(19) United States
(12) Patent Application Publication
(10) Pub. No.: US 2010/0297156 A1
(43) Pub. Date: Nov. 25, 2010

(54) ANALOGUES OF PHOSPHATIDYLINOSITOL MANNOSIDES

(75) Inventors: Gary David Ainge, Lower Hutt (NZ); Bryce Malcolm Buddle, Palmerston North (NZ); Michel Denis, Palmerston North (NZ); Robyn Louise Wright, Wellington (NZ); Darren Gibson, Wellington (NZ); Ahivali Ashwin Gulab, Wellington (NZ); David Samual Larsen, Dunedin (NZ); Gavin Frank Painter, Lower Hutt (NZ); Natalie Anne Parlane, Palmerston North (NZ)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: INDUSTRIAL RESEARCH LIMITED, Lower Hutt (NZ)

(21) Appl. No.: 12/519,253

(22) PCT Filed: Dec. 20, 2007
(86) PCT No.: PCT/NZ2007/000378

§ 371 Date: May 3, 2010

(30) Foreign Application Priority Data
Dec. 20, 2006 (NZ) 552205

(51) Int. Cl. A61K 31/7034; C07H 15/207; A61K 39/00; A61P 31/00; A61P 37/08; A61P 35/00; A61P 31/04; A61P 31/06; A61P 17/00; A61P 17/08

(52) U.S. Cl. 424/184.1; 536/17.1; 536/18.2; 514/35

(57) ABSTRACT

The invention relates to compounds which are immunomodulatory compounds and, in particular, can induce IL-12 secretion. The invention also relates to compositions containing the compounds, precursors, and prodrugs of these compounds, use of these compounds as adjuvants in combination with vaccines, and use of these compounds for treatment of diseases or conditions relating to infection, atopic disorders, or cancer.

### FIELD OF INVENTION

This invention relates generally to certain phosphatidylinositol mannoside (PIM) analogues, precursors and prodrugs of these compounds, compositions comprising these compounds, including pharmaceutical compositions and adjuvant compositions, processes for preparing the compounds, and methods of treating or preventing diseases or conditions using such compounds, especially diseases or conditions relating to infection, atopic disorders, or cancer.

### BACKGROUND

Phosphatidylinositol mannoside (PIM) is a component of mycobacterial cell walls that possesses immunomodulatory properties (Goren M. B., Am. Rev. Respir. Dis., 1982; 125:50-69). Much of the stimulatory activity of mycobacteria reside in the cell walls of these microbes, with various products such as lipoarabinomannan (LAM), phosphatidylinositol manno-oligosaccharides (PIMs), and a host of other mycobacterial compounds which modulate the immune response (Briken V., Porcelli S. A., Besra G. S., Kremer L., Mol. Microbiol. 2004; 53:391-403). The immunomodulatory properties of these products have been exploited to produce formulations with adjuvant properties (Ivanyi J., Sharp K., Jackett P., Bothamley G., Springer Semis. Immunopathol., 1988; 10:279-300). Moreover, a PIM fraction from Mycobacterium bovis was shown to possess adjuvant activity (Sprott G. D., Dicaire C. J., Gurnani K., Sad S., Krishnan., Infection and Immunity 2004; 72:5235-5246).

An antigen isolated from Mycobacterium bovis was shown to induce IFN-γ release in CD1d restricted manner and has been identified as phoshatidylinositol mannoside (PIM) (Fischer, K., Scotet, E., Niemeyer, M., Koebernick, H., Zerrahn, J., Maillet, S., Hurwitz, R., Kursar, M., Bonneville, M., Kaufmann, S. H., Schaible, U. E., Proc. Natl. Acad. Sci. USA, 2004, 101:10685). CD1 receptors are a family of lipid-antigen presenting molecules that are structurally related to the peptide-presenting molecule MHC class I molecules. However, in contrast to MHC molecules, the CD1 proteins feature a deep hydrophobic binding groove that is suited for binding hydrophobic molecules (Moody, D. B., Zajonc, D. M., Wilson I. A., Nat. Rev. Immunol. 5: 387-399). CD1 molecules are recognized by conventional T-cells and invariant natural killer T cells (iNKT cells). After stimulation, iNKT cells can modulate the function of a number of immune cells including T cells, B cells, natural killer (NK) cells and dendritic cells (DC) (Hermans, I. F., Silk, J. D., Gileadi, U., Salio, M., Mathew, B., Ritter, G., Schmidt, R., Harris, A. L., Old, L., Cerundolo, V., J. Immunol. 2003, 171:5140-5147) primarily through the release of a spectrum of cytokines, including ‘Th1’ cytokines such as IFN-γ and IL-12, and ‘Th2’ cytokines such as IL-4 and IL-13. The release of Th1 cytokines is likely to contribute to antitumour and antimicrobial functions (Smyth, M. J., Crowe, N. Y., Pellicci, D. G., Kyparissoudis, K., Kelly, J. M., Takeda, K., Yagita, H., Godfrey, D. I., Blood 2002, 99, 1259-1266; and Gonzalez-Aseguinolaza, G., Van Kaer, L., Bergmann, C. C., Wilson, J. M., Schmieg, J., Kronenberg, M., Nakayama, T., Taniguchi, M., Koezuka, Y., Tsuji, M. J. Exp. Med. 2002; 195:617-624) whereas the release of Th2 cytokines may attenuate autoimmune diseases (Miyamoto, K., Miyake, S., Yamamura, T., Nature 2001; 413:531-534). Purified PIMs have also been implicated in Granuloma formation and the recruitment of iNKT cells (Gilleron, M., Ronet, C., Mempel, M., Monsarrat, B., Gachelin, G., Puzo, G., J. Biol. Chem. 2001; 276:34896) and PIM is known to bind CD1 resulting in the activation and expansion of T-cell populations (Ernst, W. A., Maher, J., Cho, S., Niazi, K. R., Chatterjee, D., Moody, D. B., Besra, G. S., Watanabe, Y., Jensen, P. E., Porcelli, S. A., Kronenberg, M., Modlin, R. L., Immunity 1998; 8:331-40).

A general structure for the natural PIM molecule as isolated from mycobacteria comprises a diacylglycerol unit, linked to a D-myo-inositol group through a phosphodiester group. The inositol group is glycosylated on O-2 and O-6 with mannopyranose units as depicted below:

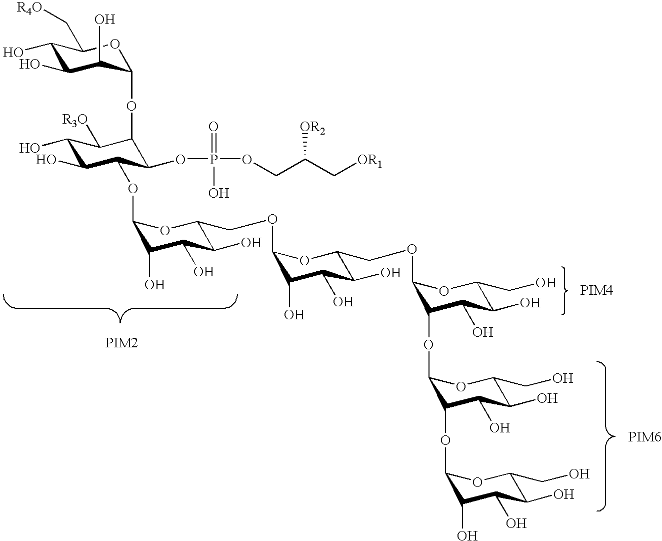

The groups R₁ and R₂ are acyl and typically comprise palmitic, stearic and tuberculostearic acids (Gilleron M., Quesniaux V. F., Puzo G., J. Biol. Chem. 2003; 278:29880). R₃ and R₄ can be H or as above for R₁ and R₂. PIM2 has a single mannopyranose at O-6, PIM4 has three mannopyranose units, and PIM6 has five mannopyranose units. The position and configurations of the glycosidic linkages have been confirmed by Severn et al. (Severn, W. B., Furneaux, R. H., Falshaw R., Atkinson, P. H., Carbohydr. Res. 1998; 308:397).

Synthetic samples of discrete PIM molecules are known to possess immunomodulating properties. For example, PIM2 and PIM1's were shown to be effective in the suppression of airways eosinophilia in an in vivo model (Ainge G. D., Hudson J., Larsen D. S., Painter G. F., Gill G. S., Harper J. L., Bioorg. Med. Chem., 2006; 14:5632-5642). Synthetic samples of PIM2 and PIM4 were also shown to be good IFN-γ inducers in an in vivo transgenic mouse model (Ainge G. D., Parlane N. A., Denis M., Hayman C. M., Larsen D. S., Painter G. F., Bioorg. Med. Chem., 2006: 14:7615-7624).

IL-12 is a potent proinflammatory Th1 cytokine which is essential for resistance to bacterial, fungal, and parasitic infections and is closely linked to IFN-γ release (for review, see, Holland and Frei, Eds., CANCER MEDICINE 6, 2003, BC Decker Hamilton, Ontario, Canada). It is produced within a few hours of infection, activates NK cells and, through its ability to induce IFN-γ production, enhances the phagocytic and bacteriocidal activity of phagocytic cells and their ability to release proinflammatory cytokines, including IL-12 itself. IL-12 is also a key immunoregulatory molecule, especially of Th1 responses (Hisieh C., et al., Science 1993; 260:547-9). IL-12 allows differentiation and function of the Th1 T cells, and inhibits differentiation of Th2 T cells (see, e.g., Holland and Frei, supra).

IL-12 is produced primarily by phagocytic cells (Trinchieri G., Scott P., Curr. Top. Microbiol. Immunol. 1999; 238:57-78; Jacobsen S. E. W., Res. Immunol. 1995; 146:506-14) and sets the stage for antigen-specific immune response. EBV-transformed B cell lines constitutively produce at least low levels of IL-12 p40, as do malignant B cells. However, the relation of IL-12 production to normal B cells remains to be established. Resting T and NK cells do not express IL-12R or express it only at very low levels. However, resting peripheral blood T and NK cells rapidly respond to IL-12 with IFN-γ production and enhancement of cytotoxic functions, suggesting that the receptor is present, at least in a proportion of the cells, and/or it can be rapidly activated in culture (see, e.g., Holland and Frei, supra).

IL-12 is a heterodimeric cytokine, composed of a heavy chain of 40 kD (p40) (IL-12B) and a light chain of 35 kD (p35) (IL-12A) (Trinchieri G., Scott P., supra). The two chains are most closely related to gpl 30. The gene encoding the p35 light chain has limited homology with single-chain cytokines, whereas the gene encoding the p40 heavy chain is homologous to extracellular domains of haematopoietic cytokine receptors (see, e.g., Holland and Frei, supra). A positive feedback mechanism exists between IL-12 and IFN-γ, with each of these molecules acting as potent inducers of the other. This amplification loop can be modified by IL-10, TGF-β, IL-4, and IL-13, which doWnregulate IL-12 production and the ability of T and NK cells to respond to IL-12. Th2 cells, by producing IL-4, and IL-13, suppress IL-12 production and prevent the emergence of a Th1 response (see, e.g., Holland and Frei, supra).

Because of the central role of IL-12 in diseases and other conditions involving immune responses and immunoregulation, it is of key importance to identify agents which can modify IL-12 production and/or levels in a selective manner.

IL-10 is an anti-inflammatory or regulatory cytokine which diminishes the release of IL-12, and the balance in the production of these factors will determine the direction of the immune response. Products with adjuvant activities for cell-mediated immune responses are expected to stimulate IL-12, but some IL-10 release prevents the adjuvants from having highly toxic side-effects associated with an unchecked release of IL-12 (Martin M., Michalek S. M., Katz J., Infect Immun. 2003; 71:2498-507 and Persing D. H., Coler R. N., Lacy M. J., Johnson D. A., Saldridge J. R., Hershberg R. M., Reed S. G., Trends Microbiol. 2002; 10:S32-7).

The invention relates to phosphatidylinositol mannoside (PIM) analogues having at least one ether linkage replacing an acyl linkage. Such compounds have been identified as surprisingly potent agents for modifying immune responses and immunoregulation, for example, by inducing IL-12 secretion. The compounds of the invention include those of the formulas as set forth below.

It is therefore an object of the invention to provide phosphatidylinositol mannoside analogues useful as agents for treating diseases or conditions relating to infection, atopic disorders, or cancer, or to at least provide a useful alternative.

### STATEMENTS OF INVENTION

In a first aspect, the invention relates to a compound of the formula (I):

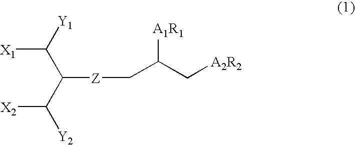

where:
X₁ and X₂ are H, or taken together form a 6-membered carbocyclic ring which is optionally substituted with one or more groups selected from OH, halogen, and NH₂;
Y₁ and Y₂ are independently H, OH, or a saccharide having 1 to 5 glycosyl or glycosyloxy units, provided that Y₁ and Y₂ are not both H;
Z is —O—(CH₂)ₙ₌₁₋₆—O—, —O——C(═O)—O—, —NH—C(═O)—O—, —O—C(═O)—NH—, —O—P(OH)(═O)—O—, —CH₂—P(—OH)(═O)—O—, —O—P(OH)(═O)—CH₂—, —O—P(OH)(═S)—O—, —CH₂—P(—OH)(═S)—O—, —O—P(—OH)(═S)—CH₂—, —CF₂—P(—OH)(═O)—O—, —CHF—P(—OH)(═O)—O—, —O—P(—OH)(═O)—CF₂, or —O—P(—OH)(═O)—CHF—;
A₁ and A₂ are independently O, NH, CH₂, CHF, CF₂; and
R₁ and R₂ are independently linear or branched alkyl or acyl groups having up to 30 carbon atoms, which may be saturated or may be unsaturated having up to 4 units of unsaturation, and provided that R₁ and R₂ are not both acyl;
   or a pharmaceutically acceptable salt or hydrate thereof.

Preferably X₁ and X₂ are both H. Alternatively, X₁ and X₂ taken together form a 6-membered carbocyclic ring.

It is preferred that the 6-membered carbocyclic ring formed by X₁ and X₂ is an inositol. In certain embodiments of the invention, one or more of the secondary hydroxyl groups of the inositol are replaced with alkyloxy or acyloxy groups.

Preferably the inositol is D-myo-inositol. The 3-hydroxyl group of the D-myo-inositol may be absent, or the 3-hydroxyl group may be replaced with an alkyloxy group or an acyloxy group. The alkyloxy group preferably has the formula —OCₙH₂ₙ₊₁, where n=6 to 30, especially 14 to 26. The acyloxy group preferably has the formula —OC(═O)—CₙH₂ₙ₊₁, where n=6 to 30, especially 14 to 26. Where the alkyl or acyl groups are unsaturated, they preferably include up to four units of unsaturation.

Preferably at least one of Y₁ and Y₂ is a saccharide having 1 to 5 glycosyl or glycosyloxy units. It is further preferred that both of Y₁ and Y₂ is a saccharide having 1 to 5 glycosyl or glycosyloxy units. The glycosyl or glycosyloxy units may be attached by a glycosidic linkages or by β glycosidic linkages. It is further preferred that the glycosyl or glycosyloxy units are attached by 1-6α glycosidic linkages and/or 1-2α glycosidic linkages.

In preferred embodiments, the glycosyl or glycosyloxy units are each independently selected from mannosyl, mannosyloxy, galactosyl, galactosyloxy, glucosyl, glucosyloxy, glucosaminyl, and glucosaminyloxy. The glycosyl or glycosyloxy units may be D- or L-isomers, but are preferably D-isomers.

In other embodiments, one or more of the hydroxyl groups of one or more of the glycosyl or glycosyloxy units are replaced with an alkyloxy group or an acyloxy group. The alkyloxy group preferably has the formula CₙH₂ₙ₊₁, where n=6 to 30, especially 14 to 26. The acyloxy group preferably has the formula —OC(═O)CₙH₂ₙ₊₁, where n=6 to 30, especially 14 to 26. Where the alkyloxy and acyloxy groups are unsaturated, they preferably include up to four units of unsaturation.

In a further aspect, the invention relates to a compound of the formula (2):

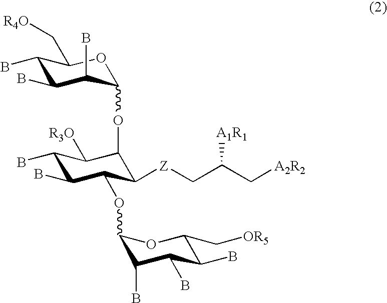

where:
A₁, A₂, R₁, R₂, and Z are as defined above;
R₃ and R₄ are each independently H, or linear or branched alkyl or acyl groups having up to 30 carbon atoms, which maybe saturated or may be unsaturated having up to 4 units of unsaturation;

R₅ is H or a saccharide having 1 to 4 glycosyl or glycosyloxy units, where each glycosyl or glycosyloxy unit is selected from mannosyl, mannosyloxy, galactosyl, galactosyloxy, glucosyl, glucosyloxy, glucosaminyl, and glucosaminyloxy;
each B is independently H or OH; and
   where the compound can include α and/or β glycosidic linkages.

Each alkyl group in formula (2) preferably has the formula CₙH₂ₙ₊₁, where n=6 to 30, especially 14 to 26. Each acyl group in formula (2) preferably has the formula C(═O)CₙH₂ₙ₊₁, where n=6 to 29, especially 14 to 26. Where the alkyl or acyl groups in formula (2) are unsaturated, they preferably include up to four units of unsaturation.

Preferably R₁ and R₂ are each independently C₈H₁₇, C₁₆F¹3₃, C₂₆H₅₃, COC₈H₁₇, COC₁₅H₃₁, COC₂₅H₅₁, or COC₂₆H₅₃; provided R₁ and R₂ are not both COC₈H₁₇, COC₁₅H₃₁, COC₂₅H₅₁, or COC₂₆H₅₃.

Preferably R₃, and R₄ are each independently H, C₈H₁₇, C₁₆H₃₃, C₂₆H₅₃, COC₈H₁₇, COC₁₅H₃₁, COC₂₅H₅₁, or COC₂₆H₅₃.

A₁ and A₂ are preferably 0.

B is preferably OH.

R₅ is preferably H.

Specific compounds of the invention include:

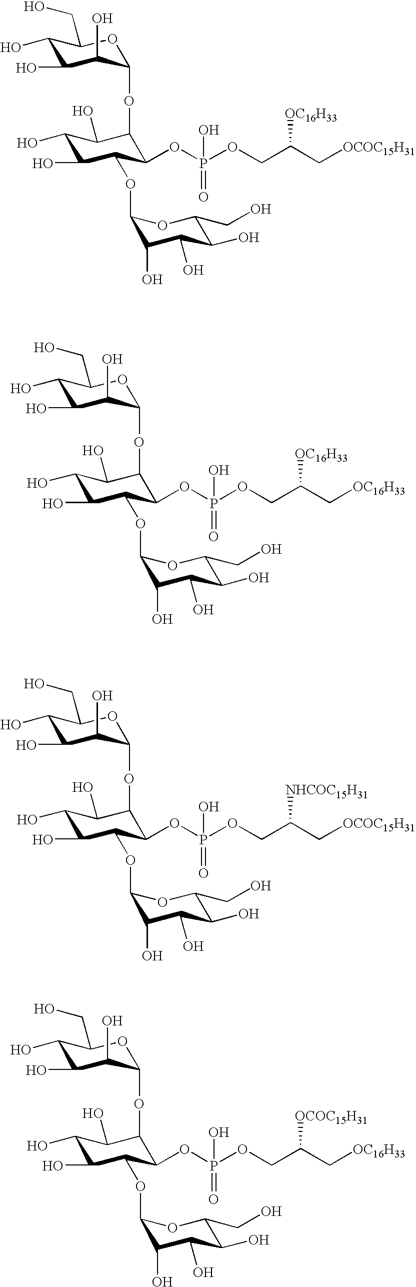

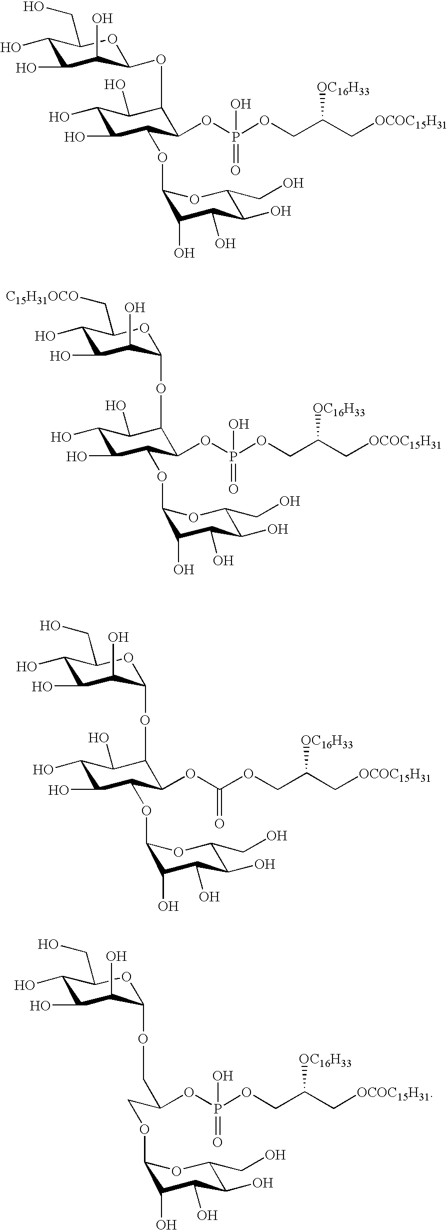

In a further aspect, the invention relates to a compound of the formula (3):

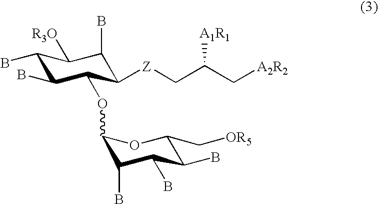

where A₁, A₂, R₁, R₂, R₃, R₅, B and Z are as defined above; and where the compound can include α and/or β glycosidic linkages.

Each alkyl group in formula (3) preferably has the formula CₙH₂ₙ₊₁, where n=6 to 30, especially 14 to 26. Each acyl group in formula (3) preferably has the formula CO—CₙH₂ₙ₊₁, where n=6 to 29, especially 14 to 26. Where the alkyl and acyl groups in formula (3) are unsaturated, they preferably include up to four units of unsaturation.

Preferably, R₁ and R₂ are each independently C₈H₁₇, C₁₆H₃₃, C₂₆H₅₃, COC₈H₁₇, COC₁₅H₃₁, COC₂₅H₅₁, or COC₂₆H₅₃; provided R₁ and R₂ are not both COC₈H₁₇, COC₁₅H₃₁, COC₂₅H₅₁, or COC₂₆H₅₃.

Preferably R₃ is H, C₈H₁₇, C₁₆H₃₃, C₂₆H₅₃, COC₈H₁₇, COC₁₅H₃₁, COC₂₅H₅₁, or COC₂₆H₅₃.

A₁ and A₂ are preferably 0.

B is preferably OH.

R₅ is preferably H.

Specific compounds of this aspect of the invention include:

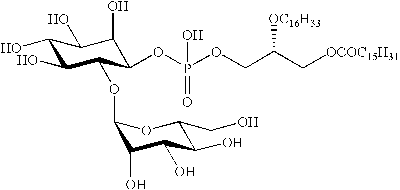

In a further aspect, the invention relates to a compound of the formula (4):

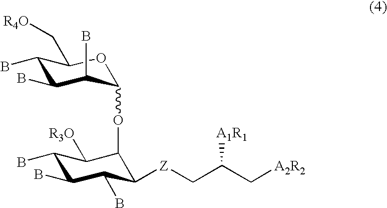

where A₁, A₂, R₁, R₂, R₃, R₄, B, and Z are as defined above; and where the compound can include α and/or β glycosidic linkages.

Each alkyl group in formula (4) preferably has the formula CₙH₂ₙ₊₁, where n=6 to 30, especially 14 to 26. Each acyl group in formula (4) preferably has the formula CO—CₙH₂ₙ₊₁, where n=6 to 29, especially 14 to 26. Where the alkyl and acyl groups in formula (4) are unsaturated, they preferably include up to four units of unsaturation.

Preferably R₁ and R₂ are each independently C₈H₁₇, C₁₆H₃₃, C₂₆H₅₃, COC₈H₁₇, COC₁₅^{H}₃₁, COC₂₅H₅₁, or COC₂₆H₅₃; provided R₁ and R₂ are not both COC₈H₁₇, COC₁₅H₃₁, COC₂₅H₅₁, or COC₂₆H₅₃.

Preferably R₃, and R₄ are each independently H, C₈H₁₇, C₁₆H₃₃, C₂₆H₅₃, COC₈H₁₇, COC₁₅H₃₁, COC₂₅H₅₁, or COC₂₆H₅₃.

A₁ and A₂ are preferably 0.

B is preferably OH.

Specific compounds of this aspect of the invention include:

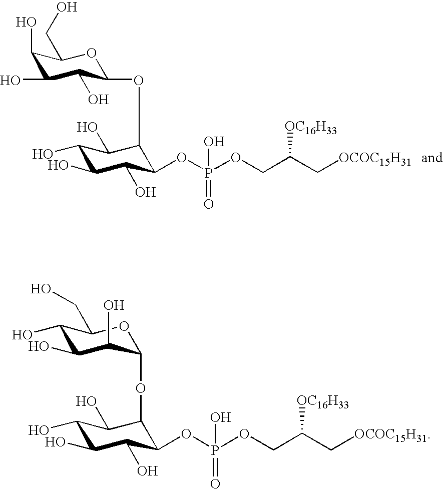

In yet a further aspect, the invention relates to a compound of the formula (5):

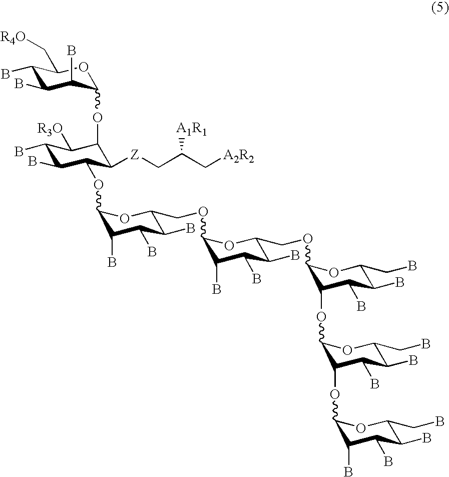

where A₁, A₂, R₁, R₂, R₃, R₄, B, and Z are as defined above; and where the compound can include α and/or β glycosidic linkages.

Each alkyl group in formula (5) preferably has the formula CₙH₂ₙ₊₁, where n=6 to 30, especially 14 to 26. Each acyl group in formula (5) preferably has the formula CO—CₙH₂ₙ₊₁, where n=6 to 29, especially 14 to 26. Where the alkyl and acyl groups in formula (5) are unsaturated, they preferably include up to four units of unsaturation.

Preferably R₁ and R₂ are each independently C₈H₁₇, C₁₆H₃₃, C₂₆H₅₃, COC₈H₁₇, COC₁₅H₃₁, COC₂₅H₃₁, or COC₂₆H₅₃; provided R₁ and R₂ are not both COC₈H₁₇, COC₁₅H₃₁, COC₂₅H₅₁, or COC₂₆H₅₃.

Preferably R₃, and R₄ are each independently H, C₈H₁₇, C₁₆H₃₃, C₂₆H₅₃, COC₈H₁₇, COO₁₅H₃₁, COC₂₅H₅₁, or COC₂₆H₅₃.

A₁ and A₂ are preferably O.

B is preferably OH.

In a further aspect, the invention relates to compositions of the compounds of the invention. In particular, pharmaceutical compositions and adjuvant compositions, which contain a compound of the invention, or a pharmaceutically acceptable salt or hydrate thereof, and one or more pharmaceutically acceptable carriers, excipients, or diluents.

The pharmaceutical compositions can be specifically formulated for oral, intravenous, inhalation, subcutaneous, or intranasal delivery. Also related are prodrugs comprising these compounds or pharmaceutical compositions thereof. The invention also provides pharmaceutical compositions which are adjuvant compositions including, for example, vaccines.

The invention further relates to methods of treatment or prevention employing the compounds of the invention or compositions comprising these compounds. The invention also provides the use of the compounds in the preparation of a medicament for treatment of a patient. Specifically set out for treatment or prevention are conditions or diseases relating to infection, atopic disorders, or cancer.

Other aspects and embodiments of the invention are described in detail herein.

### BRIEF DESCRIPTION OF FIGURES

This invention is described with reference to specific embodiments thereof and with reference to the figures:

FIG. 1: Induction of IL-12 by compounds on bovine dendritic cells (DC) (50 μg of each compound).

FIG. 2: Induction of IL-10 and IL-12 by compounds on mouse dendritic cells (DC) (50 μg of each compound).

### DETAILED DESCRIPTION

Definitions

The term “alkyl” means any saturated or unsaturated hydrocarbon radical, and includes any C₁-C₂₅, C₁-C₂₀, C₁-C₁₅, C₁-C₁₀, or C₁-C₆ alkyl group, and is intended to include both straight- and branched-chain alkyl groups. Examples of alkyl groups include, but are not limited to: methyl, ethyl, n-propyl, iso-propyl, n-butyl, iso-butyl, sec-butyl, t-butyl, n-pentyl, decyl, dodecyl, hexadecyl, pentacosyl, and (R)- or (S)-9-methylhexadecyl.

The term “acyl” means a carbonyl group (—C═O) attached to an alkyl group, i.e. —CO-alkyl.

The term “alkoxy” means an hydroxy group with the hydrogen replaced by an alkyl group, i.e. —O-alkyl.

The term “acyloxy” means an hydroxy group with the hydrogen replaced by an acyl group, i.e. —O—CO-alkyl.

The term “halogen” includes fluorine, chlorine, bromine, and iodine.

The term “glycosyl” means a moiety obtained by removing the hydroxyl group from the hemiacetal function (the anomeric carbon) of a saccharide, and includes glucosyl, mannosyl, galactosyl, glucosaminyl, etc.

The term “saccharide” means a carbohydrate or sugar moiety having one or more glycosyl or glycosyloxy moieties, and includes monosaccharides, oligosaccharides, and polysaccharides.

The term “inositol” means a six-membered carbocyclic ring where each carbon atom of the ring is hydroxylated. For example, D-myo-inositol is:

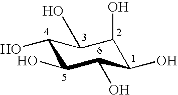

### and D-chiro-inositol is:

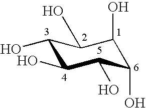

The term “cancer” and like terms refer to a disease or condition in a patient that is typically characterized by abnormal or unregulated cell growth. Cancer and cancer pathology can be associated, for example, with metastasis, interference with the normal functioning of neighbouring cells, release of cytokines or other secretory products at abnormal levels, cell proliferation, tumour formation or growth, suppression or aggravation of inflammatory or immunological response, neoplasia, premalignancy, malignancy, invasion of surrounding or distant tissues or organs, such as lymph nodes, etc. Particular cancers are described in detail herein.

“Infections” and like terms refer to diseases or conditions of a patient comprising internal and/or external growth or establishment of microbes. Microbes include all living forms too small to be seen by eye, including bacteria, viruses, fungi, and protozoa. Included are aerobic and anaerobic bacterial, and gram positive and gram negative bacteria such as cocci, bacilli, spirochetes, and mycobacteria. Particular infectious disorders are described in detail herein.

“Atopic disorders” and like terms refer to a disease or condition of a patient that is typically characterized by an abnormal or upregulated immune response, for example, an IgE-mediated immune response, and/or Th2-cell immune response. This can include hypersensitivity reactions (e.g., Type I hypersensitivity), in particular, as associated with allergic rhinitis, allergic conjunctivitis, atopic dermatitis, and allergic (e.g., extrinsic) asthma. Typically, atopic disorders are associated with one or more of rhinorrhea, sneezing, nasal congestion (upper respiratory tract), wheezing, dyspnea (lower respiratory tract), itching (e.g., eyes, skin), nasal turbinate edema, sinus pain on palpation, conjunctival hyperemia and edema, skin lichenification, stridor, hypotension, and anaphylaxis. Particular atopic disorders are described in detail herein.

The term “prodrug” as used herein means a pharmacologically acceptable derivative of a compound of any one of formulas disclosed herein, such that an in vivo biotransformation of the derivative gives the compound as defined in any one of the formulas. Prodrugs of compounds of these formulas may be prepared by modifying functional groups present in the compounds in such a way that the modifications are cleaved in vivo to give the parent compound.

The term “unsaturation” means a carbon-carbon double bond or triple bond. For example, 4 units of unsaturation means 4 carbon-carbon double or triple bonds.

The term “pharmaceutically acceptable salt” is intended to apply to non-toxic salts derived from inorganic or organic acids, including, for example, the following acid salts: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, p-toluenesulfonate, salicylate, succinate, sulfate, tartrate, thiocyanate, and undecanoate.

The term “patient” includes human and non-human animals. Non-human animals include, but are not limited to birds and mammals, in particular, mice, rabbits, cats, dogs, pigs, sheep, goats, cows, horses, and possums.

“Treatment” and like terms refer to methods and compositions to prevent, cure, or ameliorate a medical disease, disorder, or condition, and/or reduce at least a symptom of such disease or disbrder. In particular, this includes methods and compositions to prevent or delay onset of a medical disease, disorder, or condition; to cure, correct, reduce, slow, or ameliorate the physical or developmental effects of a medical disease, disorder, or condition; and/or to prevent, end, reduce, or ameliorate the pain or suffering caused the medical disease, disorder, or condition.

For the purposes of the invention, any reference to the disclosed compounds includes all possible formulations, configurations, and conformations, for example, in free form (e.g., as a free acid or base), in the form of salts or hydrates, in the form of isomers (e.g., cis/trans isomers), stereoisomers such as enantiomers, diastereomers, and epimers, in the form of mixtures of enantiomers or diastereomers, in the form of racemates or racemic mixtures, or in the form of individual enantiomers or diastereomers. Specific forms of the compounds are described in detail herein.

Description of PIM Analogues

The invention relates to the surprising finding that compounds comprising one or more ether linkages rather than acyl linkages show potent induction of IL-12 secretion from dendritic cells (DC). The compounds of the invention include, but are not limited to, those encompassed by the formulae above, and the specific compounds shown in the Examples and in Table 1.

TABLE 1

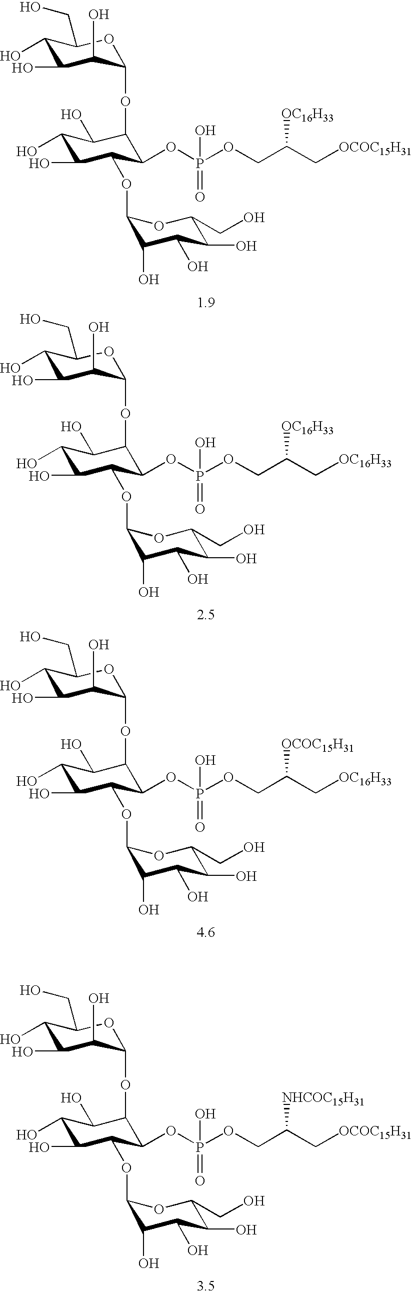

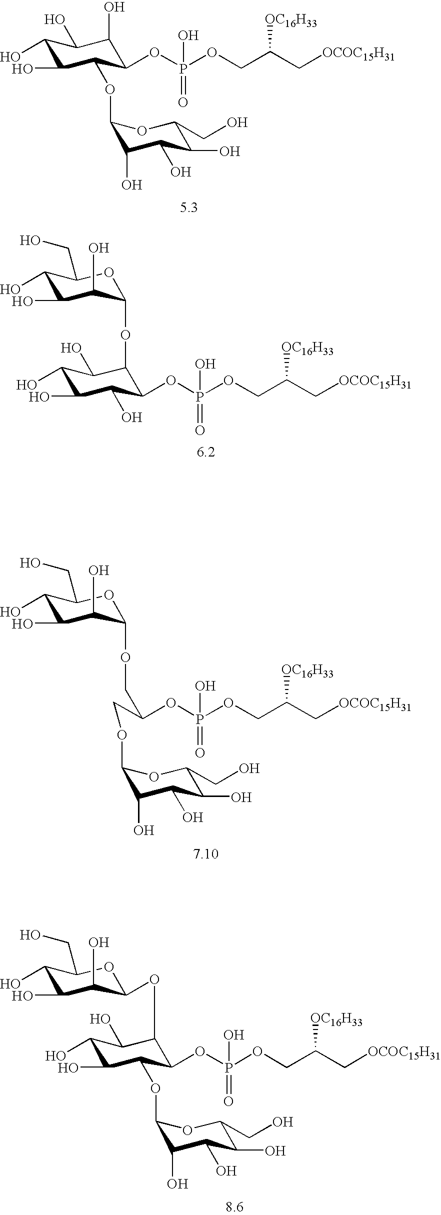

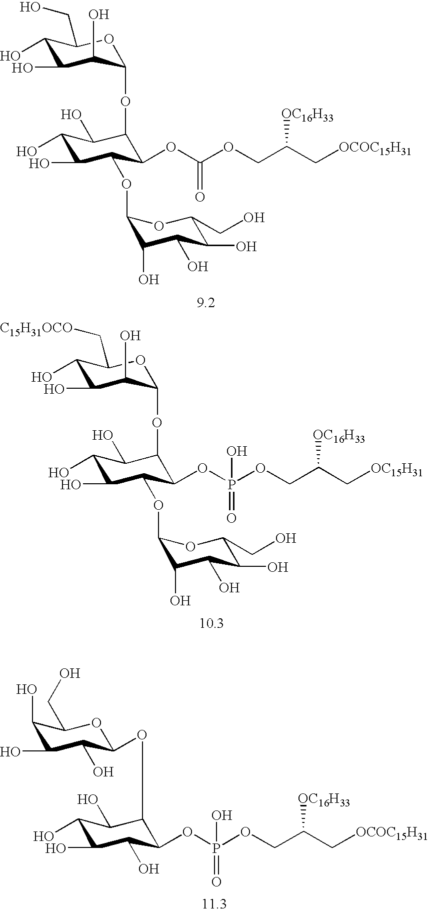

Previous PIMs and PIM analogues have been reported, for example, in WO 02/02140, WO 03/068789, and WO 2005/049631. However, the inventors are the first to demonstrate the synthesis of ether-linked PIM compounds and their unexpected induction of IL-12 from DC. Based on the knowledge of the lipid antigen presenting molecule CD1d and its interaction with PIM2 and other PIM molecules, it could not be predicted that ether-linked PIM compounds would be effective inducers of IL-12. It was previously considered that CD1d was important for the observed activity of PIM compounds and hydrogen bonding from the sn-2 carbonyl oxygen to the enzyme was an important aspect in the stability of the PIM2-CD1d complex (Zajonc D. M., Ainge G. D., Painter G. F., Severn W. B., and Wilson I. A., J. Immunology, 2006; 177:4577-4583). Also, bovine DC's have recently been shown to contain no functionally active CD1d (Rhijn I. V., Koets A. P., Im J. S., Piebes D., Reddington F., Besra G. S., Porcelli S. A., van Eden W., Rutten V. P. M. G., J. Immunology, 2006; 176:4888). However, compounds of the invention show notable activity in bovine and mouse DC (Ainge G. D., Parlane N. A., Denis M., Harer A., Hayman C. M., Larsen D. S., Painter G. F., J. Org. Chem., 2007; 72:5291-5296). Without wishing to be bound by theory, these results may indicate that hydrogen bonding to CD1d protein, or CD1d protein, itself, is not important for this activity.

Methods of Treatment

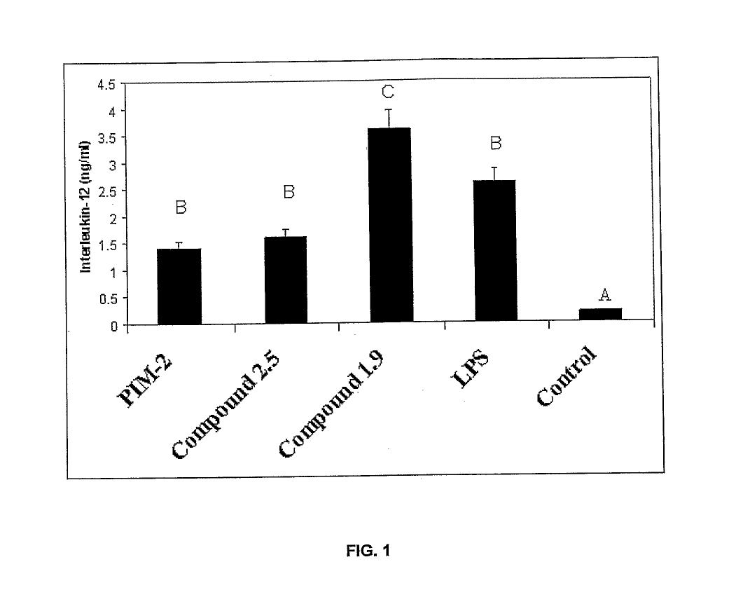

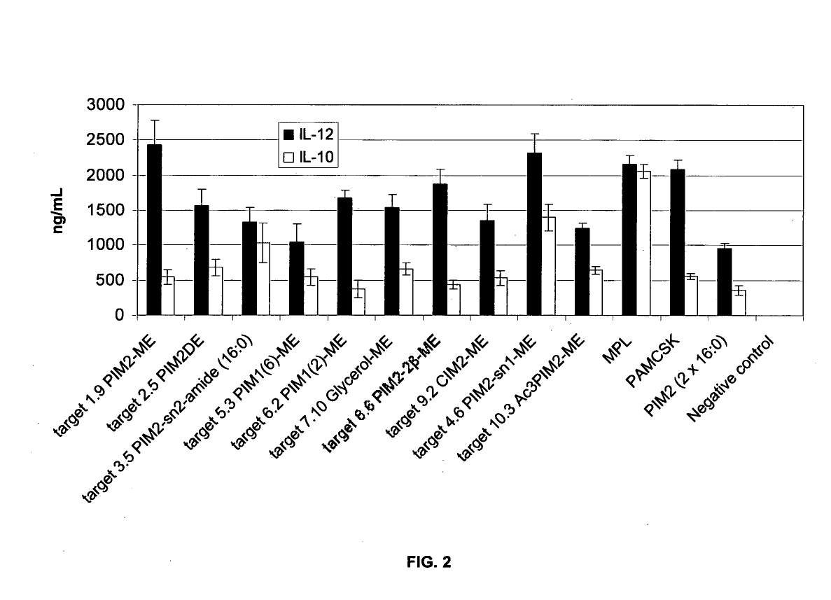

The IL-12 inducing activities of compounds 1.9 and 2.5 were tested in an in vitro bovine dendritic cell (DC) assay (FIG. 1), as described in Example 12. The IL-10 and IL-12 inducing activities of compounds 1.9, 2.5, 3.5, 4.6, 5.3, 6.2, 7.10, 8.6, 9.2, and 10.3 were tested in an in vitro mouse dendritic cell (DC) assay (FIG. 2), as described in Example 13. Positive results were obtained for both IL-10 and IL-12, but particularly for IL-12.

The central role of IL-12 in the differentiation of Th1 cells and the induction of IFN-γ indicates that the compounds of the invention could be used, in particular aspects, as a therapeutic agent designed to enhance the cellular immune response against cancer cells. As examples, the compounds can be used alone, in combination, or in conjunction with vaccine strategies. The induction of IFN-γ appears to be critical, both to the demonstrated antitumour and antimicrobial activities of IL-12 (Smyth, M. J., Crowe, N. Y., Pellicci, D. G., Kyparissoudis, K., Kelly, J. M., Takeda, K., Yagita, H., Godfrey, D. I., Blood 2002, 99, 1259-1266). In addition, IL-12 has been shown as an endogenous angiogenesis inhibitor. Initial clinical development strategies in cancer include the study of IL-12 alone in Phase I trials, followed by Phase II trials in renal cell carcinoma and other malignancies, and combination trials of IL-12 administered with cancer vaccines (Golab J., Zagozdzon R., Int. J. Mol. Med. 1999; 3:537-44; Chougnet C., Shearer G. M., Curr. Opin. Hematol. 1996; 3:216-22). Advantageously, the disclosed compounds can be used to induce production of endogenous IL-12 and obviate the need for systemic administration of this cytokine. Importantly, the disclosed compounds also cause the secretion of some IL-10 that is known to be important in the suppression of possible toxic side effects (Martin M., Michalek S. M., Katz J., Infect Immun. 2003; 71:2498-507).

In other aspects, compounds can be used to induce IL-12, and thereby potentiate the proliferation of T cells in response to various mitogens. Similarly, the compounds are also expected to produce a proliferative effect on preactivated T and NK cells and enhance the generation of cytotoxic T cells and lymphokine activated killer cells. As such, the compounds can be used to prevent or treat attacks by intracellular pathogens, especially mycobacteria and salmonellae. Specifically targeted are strains of mycobacteria, such as Mycobacterium avium and Mycobacterium bovis, as well as Mycobacterium tuberculosis. Moreover, the compounds can be used in various methods to promote survival and proliferation of early multi-potent haematopoietic progenitor cells and lineage-committed precursor cells (Jacobsen S. E. W., Res. Immunol. 1995; 146:506-14).

In additional aspects, the compounds of the invention can be used in the treatment and prevention of Th2 mediated disease, particularly asthma. In particular, the compounds are expected to suppress the allergic response which would normally cause recruitment and activation of eosinophils to the lung causing chronic swelling and inflammation of the airways that affects the breathing of sufferers. For example, experiments using a mouse model of airway eosinophilia can be used to test administration of the compounds and look for a dose dependent decrease in the number of eosinophils in the lungs of such mice (Ainge G. D., Hudson J., Larsen D. S., Painter G. F., Gill G. S., Harper J. L., Bioorg. Med. Chem., 14 (2006) 5632-5642). The in vitro cytokine profile, including IL-4, 10, 12 and IFN-γ can be measured for spleen cells incubated with compounds. See, e.g. WO 2005/049631.

The compounds can also be used for boosting the immune system or immune response in older patients (e.g., elderly or geriatrics), for example, where there are decreased levels of T cell function and/or B cell function, and increased susceptibility to infections and cancer, and reduced antibody response to immunization (Weng N. P., Immunity. 2006, 24:495-9).

For specific aspects of the invention, cancers include, but are not limited to bone, brain, breast, digestive, gastrointestinal, endocrine, eye, genitourinary, germ cell, gynaecologic, head and neck, haematologic, lung, lymphoma, musculoskeletal, neurological, respiratory, thoracic, and skin cancers, as well as AIDS-related cancers. Specifically included are bladder cancer, melanoma and non-melanoma skin cancer, breast cancer, colon and rectal cancer (e.g., colorectal cancer), pancreatic cancer, endometrial cancer, prostate cancer, kidney (renal cell) cancer, thyroid cancer, and lung cancer, and also leukaemia and Non-Hodgkin's lymphoma. Particularly included are carcinomas such as squamous cell carcinoma, adenocarcinoma, basal cell carcinoma, large cell carcinoma, renal cell carcinoma, and hepatocellular carcinoma, and sarcomas such as osteosarcoma and fibrosarcoma, and also neuroblastoma, glioma, astrocytoma, medulloblastoma, malignant melanoma, adenoma, leukemia, lymphoma, and myeloma.

For further aspects of the invention, atopic conditions include dermatitis such as contact dermatitis, atopic dermatitis, seborrheic dermatitis, nummular dermatitis, chronic dermatitis of the hands and feet, generalized exfoliative dermatitis, stasis dermatitis, and lichen simplex chronicus. Also included are conditions of rhinitis such as acute, allergic, and chronic rhinitis, for example, atrophic rhinitis, vasomotor rhinitis, hay fever (e.g., pollinosis), perennial rhinitis, allergic conjunctivitis, as well as sinusitis, and allergic eye conditions such as urticaria and uveitis, food allergies and intolerance, allergic pulmonary conditions such as anaphylaxis, and also mastocytosis, and hives (e.g., urticaria and angiodema). Additional conditions include hypersensitivity and obstructive diseases of the lung such as hypersensitivity pneumonitis, eosinophilic pneumonias, allergic bronchopulmonary aspergillosis, giant bullae, bronchitis, bronchiospasm, emphysema, asthma (e.g., allergic or extrinsic asthma), and chronic obstructive pulmonary disease.

For other aspects of the invention, infections and related conditions include pneumonia, bacteraemia, bacterial meningitis, bacterial peritonitis, urethritis, cervicitis, proctitis, pharyngitis, salpingitis, epididymitis, gastroenteritis, enteric fever, bacillary dysentery, tetanus, ghonorhea, syphilis, toxic shock syndrome, arthritis, impetigo, infective endocarditis, focal infection, pleural empyema, pleural effusion, and tuberculosis (e.g., pulmonary and extrapulmonary). Particularly included are infections by Staphylococcus strains such as S. aureus, S. pneumoniae, and S. viridans, Neisseria strains such as N. meningitidis and N. gonorrhoeae, Enterobacteriaceae strains such as Salmonella, Shigella, Escherichia, Klebsiella, Enterobacter, Serratia, Proteus, Morganella, Providencia, Yersinia, in particular E. coli and S. typhi, S. paratyphi, S. enteritidis, S. typhimurium, S. heidelberg, S. newport, S. infantis, S. agona, S. montevideo, S. saint-paul, Clostridium strains such as Clostridium perfringens, spirochete strains such as Treponema pallidum, and also Mycobacteria strains such as M. tuberculosis, M. bovis, M. avium, and M. africanum. Of particular interest are infections relating to pulmonary tuberculosis, genitourinary tuberculosis, tuberculous meningitis, miliary tuberculosis, tuberculous peritonitis, tuberculous pericarditis, tuberculous lymphadenitis, tuberculosis of bones and joints, gastrointestinal tuberculosis, and tuberculosis of the liver.

For treatment of arthritis and related conditions, the compounds can be tested in a murineJ collagen-induced arthritis model according to the method of Kakimoto, et al., (Kakimoto K., Matsukawa A., Yoshinaga M., Nakamura H., Cell Immunol., 1995; 165:26-32), in a rat collagen-induced arthritis model according to the method of Knoerzer et al., (Knoerzer D. B., Donovan M. G., Schwartz B. D., Mengle-Gaw L. J., Toxicol. Pathol. 1997; 25:13-9), in rat adjuvant arthritis model by the method of Halloran, et al., (Halloran M. M., Woods J. M., Strieter R. M., Szekanecz Z., Volin M. V., Hosaka S., Haines G. K. 3rd, Kunkel S. L., Burdick M. D., Walz A., Koch A. E., J Immunol. 1999; 162:7492-500), in a rat streptococcal cell wall-induced arthritis model according to the method of Schimmer, at al., (Schimmer Schrier D. J., Flory C. M., Laemont K. D., Tung D., Metz A. L., Friedl H. P., Conroy M. C., Warren J. S., Beck B., Ward P. A., J Immunol. 1998; 160:1466-71) or in a SCID-mouse human rheumatoid arthritis model according to the method of Oppenheimer-Marks et al. (Oppenheimer-Marks N., Brezinschek R. I., Mohamadzadeh M., Vita R., Lipsky P. E., J. Clin. Invest. 1998; 101:1261-72). For treatment of arthritis relating to Lyme disease, the compounds can be tested according to the method of Gross at al. (Gross D., Huber B. T., Steere A. C., Curr. Dir. Autoimmun. 2001; 3:94-111). For treatment of inflammatory lung conditions, the compounds can be tested in a murine immune complex-induced lung injury model according to the method of Mulligan at al., (Mulligan M. S., Jones M. L., Vaporciyan A. A., Howard M. C., Ward P. A., J Immunol. 1993; 151:5666-74), or in a rabbit chemical-induced colitis model according to the method of Bennet at al. For treatment of autoimmune diabetes, the compounds can be tested in an NOD mouse model according to the method of Hasagawa et al., (Hasegawa Y., Yokono K., Taki T., Amano K., Tominaga Y., Yoneda R., Yagi N., Maeda S., Yagita H., Okumura K., Int Immunol. 1994; 6:831-8), or in a murine streptozotocin-induced diabetes model according to the method of Herrold at al. (Herold K. C., Baumann E., Vezys V., Buckingham F., J Autoimmun. 1997; 10:17-25). The experimental models for asthma are described in detail, above.

Pharmaceutical Compositions

The compounds of the invention are useful in both free form (e.g., as a free acid or base) and in the form of salts or hydrates (e.g., pharmaceutically acceptable salts or hydrates). Salts and hydrates of the invention are preferably well tolerated and non toxic. Many examples of salts are known to those skilled in the art and are described herein. Compounds with acidic groups, e.g., phosphates or sulfates, can form salts with alkaline or alkaline earth metals such as Na, K, Mg, and Ca, and with organic amines such as triethylamine and Tris (2-hydroxyethyl) amine. Compounds with basic groups, e.g. amines, can form salts with inorganic acids such as hydrochloric acid, phosphoric acid, or sulfuric acid, or organic acids such as acetic acid, citric acid, benzoic acid, fumaric acid, or tartaric acid. Compounds with both acidic and basic groups can form internal salts. Hydrate forms are also well known in the art, including, di-, tri-, and tetrahydrates.

The compounds can also be presented as derivatives, e.g., prodrugs that can be converted in vivo or in vitro into one or more active compounds. As other derivatives, the compounds can be linked to another agent, e.g., by chemically coupling or physical association. Examples of agents include a label or reporter molecule, a supporting substrate, a carrier or transport molecule, an effector, a drug, an antibody, and an inhibitor. Agents can be covalently linked to compounds of the invention via an appropriate functional group on the compound such as a hydroxyl group, a carboxyl group or an amino group. Other derivatives include formulating the compounds with lipids, for example, in liposomes. In addition, esters can be formed between hydroxyl or carboxylic acid groups present in the compound and an appropriate carboxylic acid or alcohol reaction partner, using techniques well known in the art.

The active compounds may be administered to a patient by a variety of routes, including oral, parenteral, intravenous, topical, dermal, cutaneous, subcutaneous, intramuscular, intraocular, transepithelial, intraperitoneal, inhalation, rectal, nasal, buccal, or via an implanted reservoir. The amount of compound to be administered will vary widely according to the nature of the patient and the nature and extent of the disorder to be treated. Typically, the dosage for an adult human will be in the range of 1 to 1000 milligrams, preferably 0.1 to 100 milligrams. The specific dosage required for any particular patient will depend upon a variety of factors, including the patient's age, body weight, general health, sex, etc. For any compound, the therapeutically effective dose can be estimated initially either in cell assays, e.g., with immune cells or microbial cells, or in animal models, usually mice, rabbits, dogs, or pigs. The animal model may also be used to determine the appropriate concentration range and route of administration.

In addition to the active ingredients, these pharmaceutical compositions may contain suitable pharmaceutically acceptable auxiliary agents such as solvents, carriers, stabilizers, penetrants, excipients, and diluents. These agents can be used to facilitate processing of the active compounds into preparations which can be used phartnaceutically. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the composition may also contain suitable stabilizers or agents which increase the solubility of the compounds to allow for the preparation of highly concentrated solutions. The composition can further include penetrants appropriate to the particular barrier to be permeated are used in the formulation. Such penetrants are generally known in the art.

For intravenous, cutaneous, or subcutaneous injection, or injection at the site of affliction, the active ingredient will be in the form of a parenterally acceptable aqueous solution which is pyrogen-free and has suitable pH, isotonicity, and stability. The compounds may also be administered in a physiologically acceptable diluent such as water or saline. Those of relevant skill in the art are well able to prepare suitable solutions using, for example, solutions of the compounds or a derivative thereof, e.g., in physiological saline, or in a dispersion prepared with glycerol, liquid polyethylene glycol or oils. The diluent may comprise one or more other ingredients such as ethanol, propylene glycol, an oil, or a pharmaceutically acceptable surfactant. Liquid pharmaceutical compositions are typically formulated to have a pH between about 3.0 and 9.0, more preferably between about 4.5 and 8:5, and still more preferably between about 5.0 and 8.0. The pH of a composition can be maintained by the use of a buffer such as acetate, citrate, phosphate, succinate, Tris, or histidine, typically employed in the range from about 1 mM to 50 mM. The pH of compositions can otherwise be adjusted by using physiologically acceptable acids or bases.

For oral administration, the compounds can be formulated into solid or liquid preparations, for example tablets, capsules, powders, solutions, suspensions and dispersions. Such preparations are well known in the art as are other oral dosage regimes not listed here. In the tablet form the compounds may be formulated with conventional tablet bases such as lactose, sucrose, and corn starch, together with a binder, a disintegration agent, and a lubricant. The binder may be, for example, corn starch, or gelatin, the disintegrating agent may be potato starch or alginic acid, and the lubricant may be magnesium stearate. For oral administration in the form of capsules, diluents such as lactose and dried cornstarch may be employed. Other components such as colourings, sweeteners, or flavourings may be added. The compounds may further be administered by means of sustained release systems. For example, they may be incorporated into a slowly dissolving tablet or capsule. When aqueous suspensions are required for oral use, the active ingredient may be combined with carriers such as water and ethanol, and emulsifying agents, suspending agents and/or surfactants may be used.

The compounds may also be administered topically. For example, the compounds may be present as ingredients in lotions, creams, or gels for administration to skin or mucous membranes. These formulations may contain the active compounds suspended or dissolved in one or more pharmaceutically acceptable carriers. Carriers for topical administration of the compounds include mineral oil, liquid petrolatum, white petrolatum, propylene glycol, 2-octyldodecanol, benzyl alcohol, sorbitan monostearate, polysorbate 60, polyoxyethylene, polyoxypropylene compound, cetearyl alcohol, cetyl ester wax, emulsifying wax, and water. For any route of administration, preservatives are generally included to retard microbial growth, extend the shelf life of the compositions, and allow multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically, employed in the range of about 0.1 to 1.0% (w/v).

The pharmaceutical composition may be formulated to deliver the active compound of the present invention directly to the mucosa of the nasal passages. This may be particularly useful for treatment of rhinitis and related diseases or conditions. Preferred direct nasal mucosal delivery formulations include a nasal spray, nasal drops, cream, or ointment. For the treatment of asthma or other respiratory conditions, the pharmaceutical compositions of the present-invention may be formulated for delivery by inhalation. Generally, this will involve oral, intranasal, or pulmonary delivery. Often, inhalation by the patient will provide the motive force to deliver the active ingredient. However, respiratory administration can also involve delivery by propellant, including in the form of an aerosol generated using a jet or ultrasonic nebuliser, as will be appreciated by a skilled artisan. Further details on techniques for formulation and administration may be found in the latest edition of Remington's Pharmaceutical Sciences (Maack Publishing Co., Easton, Pa.).

In various methods, the disclosed compounds can be used with a heterogenous mixture of PIM species produced by isolating the PIM fraction from heat killed mycobacterial organisms. See, e.g., WO 02/02140 and Sayers, L, Severn, W., Scanga, C. B., Hudson, J., le Gros., G., Harper, J. L., 2004, 114, 302-309. In other methods, the compounds can further include an amino acid residue attached to the inositol ring, for example, in association with lipid rafts/caveolae. See, e.g., WO 03/068789. In addition, the disclosed compounds can be delivered in combination with at least one of allergen immunotherapies (hyposensitization or desensitization treatments), antihistamines, sympathomimetics, cromolyn, glucocorticoids, leukotriene blockers, H1 blockers such as alkylamines, ethanolamines, ethylenediamines, piperazines, phenothiazines, and piperidines, and antibiotics, especially Isoniazid, Rifampin, Streptomycin, Pyrazinamide, Ethambutol, and Capreomycin.

The compounds of the invention can also be used in adjuvant formulations, for example, for immunization of a patient. In various aspects, the adjuvants can be administered alone or in combination with a vaccine or antigenic component. The adjuvant formulations are expected to elicit both humoral and cell-mediated immunity, and can be used, for example, with vaccines based on peptides, viral and bacterial subunits, and genetically engineered antigens. Because these compositions are intended for parenteral administration, it is preferable to make up final buffered solutions used as vaccines so that the tonicity, i.e., osmolality, is essentially the same as normal physiological fluids in order to prevent post-administration swelling or rapid absorption of the composition because of differential ion concentrations between the composition and physiological fluids. It is also preferable to buffer the saline in order to maintain a pH compatible with normal physiological conditions. Also, in certain instances, it may be necessary to maintain the pH at a particular level in order to insure the stability of certain composition components such as the glycopeptides.

Any physiologically acceptable buffer may be used for the adjuvant, but phosphate buffers are preferred. Other acceptable buffers such as acetate, Tris, bicarbonate, carbonate, and the like may be used as substitutes for phosphate buffers. The pH of the aqueous component will preferably be between 6.0 and 8.0. When the adjuvant is initially prepared, unadulterated water can be used as the aqueous component of the emulsion. When the final vaccine formulation is prepared from the adjuvant, the antigenic material can be added in a buffer at an appropriate osmolality to provide the desired vaccine composition. Typically, the aqueous component employed in these compositions will be that amount necessary to bring the value of the composition to unity. That is, a quantity of aqueous component sufficient to make 100% will be mixed, with the other components listed above in order to bring the compositions to volume. In various aspects, the adjuvant formulations will be useful for both human and veterinary vaccines.

Synthesis of PIM Analogues

The compounds of the invention may be prepared by a variety of different methods. The methods include the synthesis of an orthogonally protected inositol acceptor which can be achieved from α-methylglucoside (Bender, S. L., Budhu, R. J., J. Am. Chem. Soc. 1991, 113, 9883-98854) and glycosylation with an appropriate glycosyl donor including a trichloroacetimidate donor (Wegmann, B., Schmidt, R. R. J. Carbohydr. Chem. 1987, 6, 357). Introduction of the phosphodiesfer bond can be achieved using the H-phosphonate method (Crossman, A. Jr., Brimacombe, J. S., Ferguson, M. A. J., J. Chem. Soc., Perkin Trans. 1 1997, 2769-2774). Specific examples of PIM syntheses include the use of pentenyl donors (Jayaprakash, K. N., Lu, J., Fraser-Reid, B., Bioorg. Med. Chem. Lett. 2004, 14, 3815-3819) and introduction of the diacyl glycerol moiety by way of phosphoramidite coupling methodology to prepare a AcPIM2 compound; the utilisation of acetimidate donors (Liu, X., Stocker, B. L., Seeberger, P. H., J. Am. Chem. Soc., 128 2006, 3638) to prepare AcPIM2 and AcPIM6 compounds; the use of phosphate donors to prepare PIM2 (Watanabe, Y., Yamamoto, T., Okazaki, T., Tetrahedron 1997, 53, 903, and Watanabe, Y., Yamamoto, T., Ozaki, S., J. Org. Chem. 1996, 61, 14); PIM1 compounds can also be prepared with use of trichloroacetimidate donors (Stadelmaier, A., Schmidt, R. R., Carbohydr. Res. 2003, 338, 2557); and PIM2 can be prepared with utilisation of a resolution protocol (Elie, C. J. J., Verduyn, R., Dreef, C. E., Van der Marel, G. A., Van Boom, J. H., J. Carbohydr. Chem. 1992, 11, 715).

### Examples

The examples described herein are for purposes of illustrating embodiments of the invention. It will be appreciated that the invention is not limited to these examples.

### Example 1

### Synthesis of 2,6-(Di-O-α-D-mannopyranosyl)-1-O-(1-hexadecanoyl-2-O-hexadecyl-sn-glycero-3-phosphoryl)-D-myo-inositol (1.9)

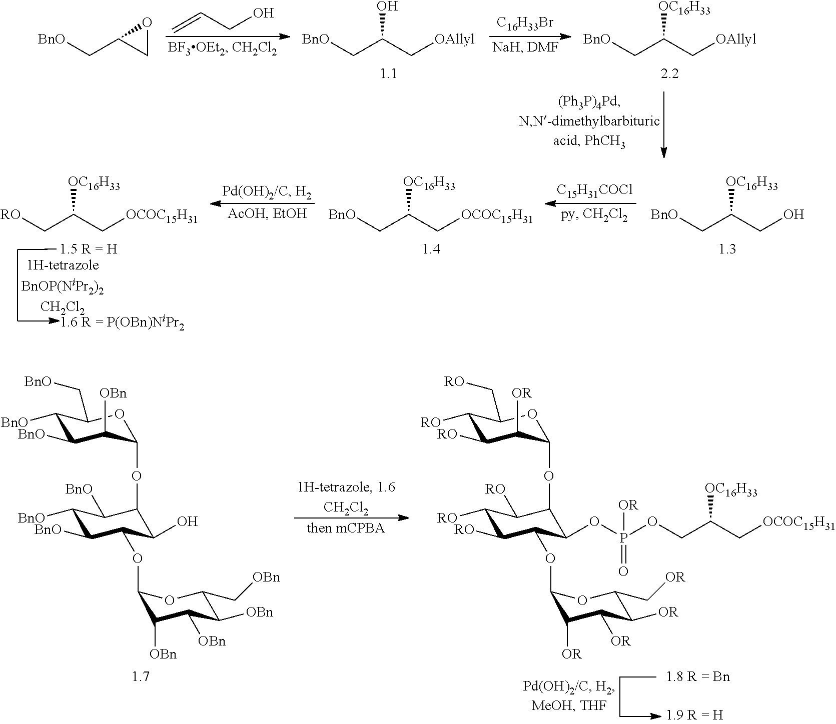

1-O-Allyl-3-O-benzyl-sn-glycerol (1.1). BF₃.OEt₂ (50 μL, 0.40 mmol) was added to a stirred solution of (R)-benzyl glycidol (261 mg, 1.60 mmol) and allyl alcohol (1.1 mL, 16 mmol) in dry CH₂Cl₂ (10 mL). After 1 h, the solvent was removed in vacuo and the residue purified by column chromatography on silica gel. Elution with EtOAc/light petroleum (1:9) afforded the title compound 1.1 (269 mg, 1.20 mmol, 76%) as a clear oil. [α]_{D}²⁸=+0.76 (c 1.00, EtOH). ¹H NMR (300 MHz, CDCl₃) δ 7.40-7.26 (m, 5H), 5.97 (ddt, J=17.3, 10.4, 5.7 Hz, 1H), 5.27 (dq, J=17.2, 1.6 Hz, 1H), 5.19 (dq, J=10.4, 1.4, 1H), 4.57 (s, 2H), 4.39-4.23 (m, 3H), 3.60-3.45 (m, 4H), 2.52 (d, J=4.4 Hz,1H). ¹³C NMR (75 MHz, CDCl₃) δ 138.0, 134.5, 128.5, 127.8, 127.8, 117.3, 73.5, 72.4, 71.4, 71.3, 69.6. HRMS-ESI [M+Na]⁺ calcd for C₁₃H₁₈O₃Na: 245.1154. Found 245.1163.

1-O-Allyl-3-O-benzyl-2-O-hexadecyl-sn-glycerol (1.2). 1-Bromohexadecane (685 μL, 2.2 mmol) was added to a stirred suspension of 1.1 (248 mg, 1.10 mmol) and sodium hydride (60% dispersion in mineral oil, 150 mg, 3.80 mmol) in dry DMF (10 mL) under nitrogen. After stirring for 16 h the reaction was quenched by addition of 1M HCl (100 mL). The mixture was extracted with CH₂Cl₂ (2×100 mL) and dried (MgSO₄). The solvent was removed in vacuo and the residue purified by column chromatography on silica gel. Elution with Et₂O/light petroleum (0:1 to 1:19) afforded the title compound 1.2 (426 mg, 0.95 mmol, 85%) as an oil. [α]_{D}²⁸=+0.60 (c 1.00, EtOH). ¹H NMR (300 MHz, CDCl₃) δ 7.36-7.24 (m, 5H), 5.89 (ddt, J=17.3, 10.2, 5.6 Hz, 1H), 5.26 (dq, J=17.2, 1.6 Hz, 1H), 5.17 (dq, J=10.4, 1.5 Hz, 1H), 4.56 (s, 2H), 4.00 (dt, J=5.6, 1.5 Hz, 2H), 3.67-3.48 (m, 7H), 1.63-1.52 (m, 2H), 1.39-1.20 (m, 26H), 0.88 (t, J=6.8 Hz, 3H). ¹³C NMR (75 MHz, CDCl₃) δ 138.5, 134.9, 128.4, 127.64, 127.57, 116.9, 78.0, 73.4, 72.4, 70.7, 70.3, 70.2, 32.0, 30.2, 29.8, 29.73, 29.70, 29.6, 29.4, 26.2, 22.8, 14.2. HRMS-ESI [M+Na]⁺ calcd for C₂₉H₅₀O₃Na: 469.3658. Found 469.3651.

3-O-Benzyl-2-O-hexadecyl-sn-glycerol (1.3). A mixture of the ether 1.2 (403 mg, 0.90 mmol), N,N′-dimethylbarbituric acid (373 mg, 2.4 mmol) and tertakis(triphenylphosphine)bailadium (61 mg, 0.050 mmol) in dry THF (4 mL) was heated at 90° C. under nitrogen in a sealed tube. After being stirred at the same temperature for 40 h the reaction mixture was cooled to rt and poured into a saturated NaHCO₃ solution and extracted with CH₂Cl₂ (2×100 mL). The organic layer was dried. (MgSO₄) and concentrated in vacuo. The residue was purified by column chromatography on silica gel. Elution with EtOAc/light petroleum (1:9) afforded the title compound 1.3 (342 mg, 0.840 mmol, 93%) as an oil. [α]_{D}²⁰=+10.8 (c 1.23, CH₂Cl₂). ¹H NMR (300 MHz, CDCl₃) δ 7.38-7.22 (m, 5H), 4.54 (m, 2H), 3.77-3.42 (m, 7H), 2.13 (t, J=5.7 Hz, 1H), 1.61-1.52 (m, 2H), 1.39-1.19 (m, 26H), 0.92-0.82 (m, 3H). ¹³C NMR (75 MHz, CDCl₃) δ 138.1, 128.5, 127.8, 127.7, 78.6, 73.6, 70.5, 70.1, 63.0, 32.0, 30.2, 29.8, 29.7, 29.5, 29.4, 26.2, 22.8, 14.2. HRMS-ESI [M+Na]⁺ calcd for C₂₆H₄₆O₃Na: 429.3345. Found 429.3351.

3-O-Benzyl-1-O-hexadecanoyl-2-O-hexadecyl-sn-glycerol (1.4). Palmitoyl chloride (110 mL, 3.64 mmol) was added dropwise to a stirred solution of alcohol 1.3 (1.34 g, 3.31 mmol) and pyridine (1.34 mL, 16.6 mmol) in CH₂Cl₂ (20 mL) cooled to 0° C. After being stirred for 12 h at rt, the reaction mixture was quenched with H₂O (100 mL). The mixture was extracted with Et₂O (2×150 mL) and the ethereal extract washed with a 0.5M HCl solution (100 mL), saturated NaHCO₃ solution (100 mL), and dried (MgSO₄). After filtration, the solvent was removed in vacuo and the residue purified by column chromatography on silica gel. Elution with EtOAc/light petroleum (0:1 to 1:9) afforded the title compound 1.4 (2.04 g, 3.18 mmol, 96%) as an oil. ¹H NMR (300 MHz, CDCl₃) δ 7.37-7.22 (m, 5H), 4.54 (m, 2H), 4.27-4.10 (m, 2H), 3.66 (quintet, J=5.2 Hz, 1H), 3.57-3.52 (m, 4H), 2.29 (t, J=7.4 Hz, 2H), 1.67-1.50 (m, 4H), 1.38-1.17 (m, 50H), 0.92-0.85 (m, 6H). ¹³C NMR (75 MHz, CDCl₃) δ 173.7, 138.2, 128.4, 127.7, 76.7, 73.5, 70.7, 69.7, 63.7, 34.3, 32.0, 30.1, 29.8, 29.7, 29.6, 29.4, 29.3, 29.2, 26.1, 25.0, 22.8, 14.2. HRMS-ESI [M+Na]⁺ calcd for C₄₂H₇₆O₄Na: 667.5641. Found 667.5632.

1-O-Hexadecanoyl-2-O-hexadecyl-sn-glycerol (1.5). A mixture of the benzyl ether 1.4 (1.00 g, 1.55 mmol) and Pd(OH)₂/C (20%, 300 mg) in EtOH (100 mL)/HOAc (10 mL) was stirred under hydrogen for 14 h. The hydrogen was removed and the mixture filtered through Celite. The filtrate was concentrated in vacuo and the residue purified by column chromatography on silica gel. Elution with EtOAc/CH₂Cl₂ (1:24) afforded the title compound 1.5 (850 mg, 1.54 mmol, 99%) as an oil. ¹H NMR (300 MHz, CDCl₃) δ 4.18-4.15(m, 2H), 3.65-3.43 (m, 5H), 2.32 (d, J=7.4 Hz, 2H), 2.09-2.02 (m, 1H), 1.65-1.52 (m, 4H), 1.36-1.21 (m, 50H), 0.92-0.85 (m, 6H). ¹³C NMR (75 MHz, CDCl₃) δ 173.8, 77.8, 70.6, 62.8, 62.8, 34.3, 32.0, 30.1, 29.8, 29.7, 29.6, 29.4, 29.3, 29.2, 26.1, 25.0, 22.8, 14.2. HRMS-ESI [M+Na]⁺ calcd for C₃₅H₇₀O₄Na: 577.5172. Found 577.5172.

Benzyl (1-O-hexadecanoyl-2-O-hexadecyl-sn-glycero)-diisopropylphosphoramidite (1.6). 1H-Tetrazole (55 mg, 0.79 mmol) was added to a stirred solution of alcohol 1.5 (395 mg, 0.714 mmol) and benzyloxy-bis-(diisopropylamino)phosphine (482 mg, 1.43 mmol) in dry CH₂Cl₂ (10 mL). After 1 h at rt, the solvent was removed in vacuo and the residue purified by column chromatography on silica gel. Elution with Et₃N/EtOAc/light petroleum (1:3:16) afforded the title compound 1.6 (544 mg, 0.689 mmol, 96%) as an oil. ¹H NMR (300 MHz, CDCl₃) δ 7.37-7.23 (m, 5H), 4.79-4.62 (m, 2H), 4.30-4.20 (m, 1H), 4.15-4.05 (m, 1H), 3.70-3.48 (m, 7H), 2.29 (t, J=7.3 Hz, 2H), 1.65-1.50 (m, 4H), 1.30-1.16 (m, 62H), 0.90-0.83 (m, 6H). ³¹P NMR (121.5 MHz, CDCl₃) δ 149.4, 1492. HRMS-ESI, [M+Na]⁺ calcd for C₄₈H₉₀NO₅NaP: 814.6454. Found 814.6469.

3,4,5-Tri-O-benzyl-2,6-di-O-(2,3,4,6-tetra-O-benzyl-α-D-mannopyranosyl)-1-O-(1-O-Hexadecanoyl-2-O-hexadecyl-sn-glycero-3-benzylphosphotyl)-D-myo-inositol (1.8). 1H-Tetrazole (10 mg, 0.14 mmol) was added to a stirred solution of 3,4,5-tri-O-benzyl-2,6-di-O-(2,3,4,6-tetra-O-benzyl-α-D-mannopyranosyl)-D-myo-inositol (1.7) (68 mg, 0.045 mmol) and phosphoramidite 1.6 (103 mg, 0.130 mmol) in dry CH₂Cl₂ (8 mL) cooled to 0° C. under argon. After stirring at rt for 2 h the reaction mixture was cooled to −40° C. and a solution of m-CPBA (50%, 60 mg, 0.19 mmol) in CH₂Cl₂ (10 mL) was transferred by cannula into the reaction mixture. After being stirred at it for 1 h the reaction was quenched by addition of a 10% Na₂SO₃ solution (50 mL) and the combined mixture extracted with Et₂O (100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9 to 1:4) followed by a second column and elution with MeOH/CH₂Cl₂ (1:50 to 1:25) afforded the title compound 1.8 (66 mg, 0.030 mmol, 67%) as an oil. ¹H NMR (300 MHz, CDCl₃) δ 7.38-7.01 (m, 60H), 5.53-5.47 (m, 1H), 5.37-5.31 (m, 1H), 5.04 (ap t, J=7.2 Hz, 2H), 4.92-4.37 (m, 21H), 4.30-3.75 (m, 17H), 3.54-3.20 (m, 9H), 2.21-2.10 (m, 2H), 1.58-1.41 (m, 4H), 1.31-1.15 (m, 50H), 0.89-0.82 (m, 6H). ¹³C (75 MHz, CDCl₃) selected signals δ 173.3, 99.6, 98.6. ³¹P NMR (121.5 MHz, CDCl₃) δ 1.17, 1.13. HRMS-ESI [M+Na]⁺ calcd for C₁₃₇H₁₇₃O₂₂NaP: 2224.2054. Found 2224.2051.

2,6-(Di-O-α-D-mannopyranosyl)-1-O-(1-O-hexadecanoyl-2-O-hexadecyl-sn-glycero-3-phosphoryl)-D-myo-inositol (1.9). Pd(OH)₂/C (20%, 25 mg) was added to a stirred solution of the fully substituted 1.8 (38 mg, 0.017 mmol) in THF/MeOH (2:3, 5 mL). The mixture was stirred under hydrogen for 2.5 h at it and the hydrogen was replaced with argon. The mixture was filtered through Celite and the filtrate concentrated in vacuo. The residue was lyophilized to afford 1.9 (19 mg, 0.16 mmol, 94%) as a white powder. [a]D=+34 (c 0.20, CHCl₃/CH₃OH/H₂O, 70:40:6). ¹H NMR (300 MHz, CDCl₃/CD₃OD/D₂O, 70:40:6) δ 5.14 (br s, 1H), 5.11 (br s, 1H), 4.50-3.20 (m, 25H), 2.35 (t, J=7.2 Hz, 2H), 1.65-1.55 (m, 4H), 1.33-1.22 (m, 50H), 0.86 (t, J=6.9 Hz, 6H). ¹³C (75 MHz, CDCl₃) δ 176.2, 103.0, 80.1, 79.8, 78.1, 74.7, 74.4, 74.2, 72.1, 71.7, 71.5, 68.4, 65.8, 65.4, 62.8, 62.6, 35.6, 33.2, 31.0, 30.7, 30.6, 27.4, 26.3, 24.0, 15.2, 10.0. ³¹P NMR (121.5 MHz, CDCl₃/CD₃OD/D₂O, 70:40:6) δ −3.5. HRMS-ESI [M−H]⁻ calcd for C₅₃H₁₀₁O₂₂P: 1119.6444. Found 1119.6453. Anal. Calcd for C₅₃H₁₀₁O₂₂P.4H₂O: C, 53.34; H, 9.21. Found: C, 53.30; H, 8.92.

### Example 2

### Synthesis of 2,6-(Di-Oα-D-mannopyranosyl)-1-O-(1,2-di-O-hexadecyl-sn-glycero-3-phosphoryl)-D-myo-inositol (2.5)

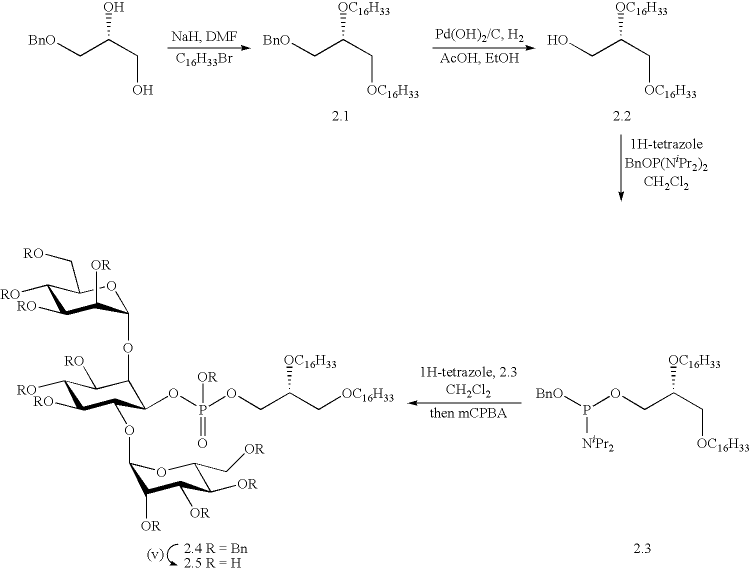

1,2-Di-O-hexadecyl-3-O-benzyl-sn-glycerol (2.1). Sodium hydride (60% dispersion in mineral oil, 500 mg, 21.1 mmol) was added to a stirred solution of 3-O-benzyl-sn-glycerol (1.61 g, 8.80 mmol) in DMF (65 mL). After 15 min bromohexadecane (6.40 mL, 21.1 mmol) was added to the reaction mixture. After being stirred at rt under an argon atmosphere for 2 h TLC indicated incomplete reaction and further NaH (60% dispersion in mineral oil, 0.2 g, 5.0 mmol) was added. After being stirred for 1 h H₂O (50 mL) was added and the mixture was extracted with Et₂O (2×50 mL). The combined organic extracts where washed with aq NaHCO₃ (sat., 2×50 mL) and aq NaCl (sat., 50 mL). After drying (MgSO₄) and filtration the solvent was removed in vacuo and the residue purified by column chromatography on silica gel. Elution with EtOAc/light . petroleum (0.5:9.5 to 1:9 to 2:8) afforded the title compound 2.1(2.37 g, 3.75 mmol, 43%) as an oil. The ¹H NMR spectrum was in good agreement with the ¹H NMR spectrum reported in literature (Bhattacharya, S., De, S., Chem. Eur. J. 1999; 5:2335-2347). HRMS-ESI (M+Na)⁺ calcd for C₄₂H₇₈O₃Na: 653.5849. Found: 653.5837.

1,2-Di-O-hexadecyl-sn-glycerol (2.2). Pd/C (10%, 237 mg) was added to a stirred solution of benzylated alcohol 2.1 (2.37 g, 3.76 mmol) in AcOH (15 mL) and EtOH (150 mL). After being stirred under a H₂ atmosphere at it for 3 h the mixture was filtered through Celite® and the filter cake washed with further EtOH (50 mL). The solvent was removed in vacuo and the residue purified by column chromatography on silica gel. Elution with EtOAc/light petroleum (0.5:9.5 to 1:9 to 2:8) afforded the title compound 2.2 (1.44 g, 2.66 mmol, 71%) as a white powder. The ¹H NMR spectrum was in good agreement with the ¹H NMR spectrum reported in literature (Browne, J. E., Freeman, R. T., Russel, J. C., Sammes, P. G., J. Chem. Soc., Perkin Trans. I 2000; 645-652). HRMS-ESI (M+Na)⁺ calcd for C₃₅H₇₂O₃Na: 563.5379. Found: 563.5352.

1,2-Di-O-hexadecyl-sn-glycero-3-O-benzyl-(N,N-dilsopropyl)-phosphoramidite (2.3). Benzyloxy-bis-(diisopropylamino) phosphine (250 mg, 0.74 mmol) in dry CH₂Cl₂ (10 mL) was transferred by cannula onto a stirred mixture of diethyl alcohol 2.2 (200 mg, 0.37 mmol) and 1H-tetrazole (26.0 mg, 70.1 mmol) in dry CH₂Cl₂ (20 mL) cooled to 0° C. The icebath was removed after 15 min and after being stirred for 2 h at it under an argon atmosphere the solvent was removed in vacuo and the residue purified by column chromatography on silica gel. Elution with Et₃N/EtOAc/light petroleum (0.3:1:9) afforded the title compound 2.3 (233 mg, 0.30 mmol, 81%) as an oil. ¹H NMR (300 MHz, CDCl₃) δ 7.37-7.25 (m, 5H), 4.79-4.63 (m, 2H), 3.68-3.40 (m, 11H), 1.57-1.53 (m, 4H), 1.25-1.17 (m, 64H), 0.88 (t, 6.43, 6H). ¹³C NMR (75 MHz, CDCl₃) δ 139.7, 128.3, 127.2, 127.0, 78.6, 71.7, 71.0, 70.7, 65.5, 65.2, 63.3, 63.1, 43.2, 43.0, 32.0, 30.2, 29.8, 29.6, 29.4, 26.2, 24.7, 24.7, 22.8, 14.2. ³¹P NMR (121.5 MHz, CDCl₃) δ 148.9 (s), 148.8(s). HRMS-ESI (M+Na)⁺ calcd for C₄₈H₉₂O₄NP: Na: 800.6662. Found: 800.6664.

3,4,5-Tri-O-benzyl-2,6-di-O-(2,3,4,6-tetra-O-benzyl-α-D-mannopyranosyl)-1-O-(2,3-di-O-hexadecyl-sn-glycerobenzylphosphoryl)-D-myo-inositol (2.4). 1H-Tetrazole (9.0 mg, 0.13 mmol) was added to a stirred solution of 1.7 (63 mg, 0.042 mmol) and phosphoramidite 2.3 (120 mg, 0.154 mmol) in CH₂Cl₂ (7 mL) at rt. After stirring at rt for 2 h under an argoh atmosphere the reaction mixture was cooled to −40° C. and a solution of pre dried m-CPBA (50%, 85.0 mg, 0.27 mmol) in CH₂Cl₂ (5 mL) was transferred by cannula into the reaction mixture. After warming to rt over 3 h the reaction was quenched by the addition of aq Na₂SO₃ (10%, 50 mL) and the mixture was extracted with Et₂O (2×30 mL). The combined organic extracts were washed with aq NaHCO₃ (sat., 3×50 mL) and aq NaCl (sat., 50 mL). After drying (MgSO₄) and filtration the solvent was removed in vacuo and the residue purified by column chromatography on silica gel. Elution with EtOAc/light petroleum (1:9 to 2:3) followed by further purification on fresh silica gel eluting with Me₂CO/toluene (3:97) afforded the title compound 2.4 (34 mg, 0.015 mmol, 36%) as an oil. 1H NMR (300 MHz, CDCl₃) δ 7.30-6.95 (m, 60H), 5.44 (d, J=7.3 Hz, 1H), 5.28 (d, J=9.7 Hz, 1H), 4.99 (t, J=7.9 Hz, 2H), 4.86-4.30 (m, 20H), 4.22 (dd, J=4.5, 12.0 Hz, 1H), 4.14-3.16 (m, 13H), 1.48 (s, 4H), 1.19 (s, 52H), 0.83-0.79 (m, 6H). ³¹P NMR (121.5 MHz, CDCl₃) δ 1.29(s), 1.25(s). HRMS-ESI (M+Na)⁺ calcd for C₁₃₇H₁₇₅O₂₁PNa: 2210.2364. Found: 2210.2314.

2,6-Di-O-(α-D-Mannopyranosyl)-1-O-(1,2-di-O-hexadecyl-sn-glycero-3-phosphoryl)-D-myo-inositol (2.5). Pd(OH)₂/C (20%, 7.0 mg) was added to a stirred solution of 2.4 (32 mg, 0.0091 mmol) in THF/MeOH (2:3, 2.5 mL). The mixture was stirred under a hydrogen atmosphere for 3 h at it when Et₃N (1 mL) was added to the mixture. The mixture was filtered through Celite® and the filter cake washed with further THF/MeOH (10 mL). The solvent was removed in vacuo and the residue was purified by colurrin chromatography on silica gel. Elution with H₂O/MeOH/CHCl₃ (0:2:7 to 0:4:7 to 0.2:4:7 to 0.8:4:7) afforded the title compound 2.5 (4.6 mg, 0.0042 mmol, 46%) as a white powder. [α]_{D}²⁰=+32 (c 0.23, D₂O/CD₃OD/CDCl₃, 0.6:4:7). ¹H NMR (300 MHz, D₂O/CD₃OD/CDCl₃, 0.5:4:7) δ 5.16 (br s, 1H), 5.11 (br s, 1H), 4.05-3.43 (m, 28H), 3.36-3.19 (m, 12H), 1.58 (br s, 5H), 1.27 (br s, 51H), 0.89 (t, J=6.5 Hz, 6H). ¹³C (75 MHz, CDCl₃) δ 101.9, 79.1, 78.1, 78.5, 77.0, 73.7, 73.3, 73.1, 72.1, 71.1, 70.6, 70.4, 67.4, 67.3, 64.9, 61.7, 61.5, 46.7, 32.2, 30.2, 30.0, 29.9, 29.6, 26.4, 26.3, 23.3, 22.9, 14.2, 9.0. ³¹P NMR (121.5 MHz, D₂O/CD₃OD/CDCl₃, 0.5:4:7) δ 0.74 (s). HRMS-ESI (M+Na)⁺ calcd for C₅₃H₁₀₃O₂₁PNa: 1129.6627. Found: 1129.6664. Microanalysis C₅₃H₁₀₃O₂₁P.4H₂O requires C 53.97% and H 9.49%. Found: C 53.71% and H 9.21%.

### Example 3

### Synthesis of 2,6-(Di-O-α-D-mannopyranosyl)-1-O-(2-deoxy-1-O-hexadeconyl-2-O-hexadeconylamino-sn-glycero-3-phosphoryl)-D-myo-inositol (3.5)

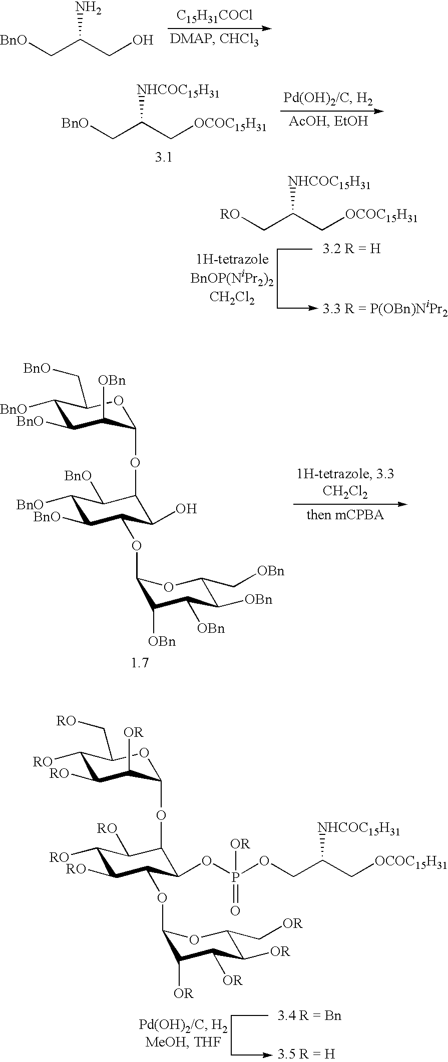

3-O-Benzyl-2-deoxy-1-O-hexadeconyl-2-hexadeconylamino-sn-glycerol (3.1). Palmitoyl chloride (1.54 mL, 5.08 mmol) was added dropwise to a stirred solution of (R)-(+)-2-amino-3-benzyloxy-1-propanol (230 mg, 1.27 mmol) and DMAP (621 mg, 5.08 mmol) in dry CHC6 (25 mL) cooled to 0° C. After stirring for 6 h at rt the reaction was quenched with the addition of H₂O (15 mL). The mixture was diluted with CHCl₃ (100 mL) and extracted with H₂O (50 ml) then 0.5 M HCl (2×50 mL). The organic layer was dried (MgSO4), filtered and the solvent removed in vacuo. The residue was purified by column chromatography on silica gel. Elution with EtOAc/light petroleum (1:4) afforded the title compound 3.1 (705 mg, 1.07 mmol, 84%) as a white solid. ¹H NMR (300 MHz, CDCl₃) δ 7.38-7.26 (m, 5H), 4.79 (d, J=8,6 Hz, 1H), 4.51 (br s, 2H), 4.42-4.31 (m, 1H), 4.25 (dd, J=10.9, 6.1 Hz, 1E);4.13 (dd, J=10.9, 6.0 Hz, 1H), 3.59 (dd, J=9.6, 3.4 Hz, 1H), 3.48 (dd, J=9.6, 4.7 Hz, 1H), 2.26 (dd, J=7.6, 7.4 Hz, 2H), 2.14 (dd, J=7.8,7.4 Hz, 2H), 1.65-1.53 (m, 4H), 1.30-1.24 (m, 48H), 0.91-0.85 (m, 6H). ¹³C NMR (75 MHz, CDCl₃) δ173.8, 172.9, 137.8, 128.5, 128.0, 127.8, 73.4, 68.8, 63.1, 48.0, 36.9, 34.3, 32.0, 29.9-29.2, 25.8, 25.0, 22.8, 14.2. HRMS-ESI [M+Na]⁺ calcd for C₄₂H₇₅NO₄Na: 680.5594. Found 680.5588.

2-Deoxy-1-O-hexadeconyl-2-O-hexadeconylamino-sn-glycerol (3.2). A mixture of benzyl ether 3.1 (663 mg, 1.01 mmol) and Pd(OH)₂/C (20%, 200 mg) in EtOH/HOAc (10:1, 33 mL) was stirred under an atmosphere of hydrogen for 16 h. The hydrogen was removed and the mixture filtered through Celite. The filtrate was concentrated in vacuo and the residue purified by chromatography on silica gel. Elution with EtOAc/light petroleum (3:7) afforded the title compound 3.2 (555 mg, 0.98 mmol, 97%) as a white solid. ¹H NMR (300 MHz, CDCl₃) δ 6.02 (d, J=6.6 Hz, 1H), 4.27-4.12 (m, 3H), 3.72-3.56 (m, 2H), 2.96 (br s, 1H), 2.33 (dd, J=7.6, 7.6 Hz, 2H), 2.19 (dd, J=7.6, 7.6 Hz, 2H), 1.67-1.55 (m, 4H), 1.35-1.19 (m, 48H), 0.91-0.84 (m, 6H). ¹³C NMR (75 MHz, CDCl₃) δ 177.6, 173.8, 62.5, 62.0, 50.5, 36.8, 34.3, 32.0, 30.0-29.2, 25.8, 25.0, 22.8. HRMS-ESI [M+Na]⁺ calcd for C₃₅H₆₉NO₄Na: 590.5124. Found 590.5118.

2-Deoxy-1-O-hexadeconyl-2-O-hexadeconylamino-sn-glycero-3-O-benzyl-(N,N-diiso-propyl)-phosphoramidite (3.3). 1H-Tetrazole (28 mg, 0.397 mmol) was added to a stirred solution of alcohol 3.2 (205 mg, 0.361 mmol) and benzyloxy-bis-(diisopropylamino)phosphine (244 mg, 0.722 mmol) in dry CH₂Cl₂ (10 mL). After 1 hr at rt the solvent was removed in vacuo and the residue purified by column chromatography on silica gel. Elution with Et₃N/EtOAc/light petroleum (1:3:16) afforded the title compound 3.3 (280 mg, 0.348 mmol, 96%) as a clear oil that solidified on standing. ¹H NMR (300 MHz, CDCl₃) δ 7.37-7.25(m, 5H), 4.89-4.58 (m, 2H), 4.38-4.25 (m, 1H), 4.22-4.04 (m, 2H), 3.87-3.56 (m, 2H), 2.28 (dd, J=7.6, 7.6 Hz, 2H), 2.12-2.04 (m, 1H), 1.94-1.87 (m, 1H), 1.64-1.45 (m, 4H), 1.33-1.17 (m, 60H), 0.91-0.85 (m, 6H). ³¹P NMR (121.5 MHz, CDCl₃) δ 150.3, 149.9. HRMS-ESI [M+Na]⁺ calcd for C₄₈H₈₉N₂O₅Na: 827.6407. Found 827.6407.

3,4,5-Tri-O-benzyl-2,6-di-O-(2,3,4,6-tetra-O-benzyl-α-D-mannopyranosyl-1-O-(2-deoxy-1-O-hexadeconyl-2-O-hexadeconylamino-sn-glycero-3-benzyl phosphoryl)-D-myo-inositol (3.4). 1H-Tetrazole (10 mg, 0.135 mmol) was added to a stirred solution of alcohol 1.7 (68 mg, 0.045 mmol) and phosphoramidite 3.3 (109 mg, 0.135 mmol) in dry CH₂Cl₂ (10 mL) at it under argon. After stirring for 1 h the reaction mixture was cooled to −40° C. and a pre-dried (MgSO₄) solution of m-CPBA (50%, 47 mg, 0.135 mmol) in CH₂Cl₂ (5 mL) was transferred by canula into the reaction mixture. The reaction was left to warm to it over 2 h then quenched by the addition of a 10% Na₂SO₃ solution (20 mL). The combined mixture was extracted with Et₂O (2×50 mL) and the ethereal extract washed with saturated NaHCO₃ solution (3×50 mL) and dried (MgSO₄). After filtration, the solvent was removed in vacuo and the residue purified by chromatography on silica gel. Elution with EtOAc/light petroleum (1:9 to 1:4) afforded the title compound 3.4 (61 mg, 0.028 mmol, 62%) as an oil. ¹H NMR (300 MHz, CDCl₃) Mixture of diastereoisomers (3:7) δ 7.48-7.03 (m, 60H), 6.54 (d, J=7.5 Hz, 0.3H), 6.40 (d, J=8.6 Hz, 0.7H), 5.48 (br s, 0.3H), 5.44 (br s, 0.7H), 5.10-5.42 (m, 21H), 4.51-4.27 (m, 5H), 4.26-3.66 (m, 15H), 3.52-3.12 (m, 6H), 2.37-2.27 (m, 1H), 2.17-2.09 (m, 1H), 2.03-1.95 (m, 2H), 1.71-1.37 (m, 4H), 1.35-1.12 (m, 48H), 0.91-0.84 (m, 6H). ¹³0 NMR (125 MHz, CDCl₃) Mixture of diastereoisomers, selected signals δ 173.8, 173.5, 173.2, 173.1, 98.8, 98.7, 98.4, 98.1. ³¹P NMR (121.5 MHz, CDCl₃) Mixture of diastereoisomers δ 0.94, −0.45. HRMS-ESI [M+Na]⁺ calcd for C₁₃₇H₁₇₂NO₅PNa: 2237.2006. Found 2237.2007.

2,6-(Di-O-α-D-mannopyranosyl)-1-O-(2-deoxy-1-O-hexadeconyl-2-O-hexadeconylamino-sn-glycero-3-phosphoryl)-D-myo-inositol (3.5). Pd(OH)₂/C (20%, 40 mg) was added to a stirred solution of per-benzylated 3.4 in THF/MeOH (2:3, 5 mL). The mixture was stirred under hydrogen for 16 h at rt. The hydrogen was removed and the mixture filtered through Celite. The filtrate was concentrated in vacuo and then redissolved in MeOH/H₂O with the aid of Et₃N. The product containing solution was pre-adsorbed onto silica and purified by column chromatography on silica gel. Elution with CHCl₃/MeOH/H₂O (7:4:0.4) gave a fraction that was lyophilized to afford 3.5 (21 mg, 0.018 mmol, 75%) as a white powder. [α]_{D}²⁰=+35 (e 0.15, CDCl₃/CD₃OD/D₂O, 4:4:1). ¹H NMR (500 MHz, CDCl₃/CD₃OD/D₂O, 4:4:1) δ 5.15 (d, J=1.6 Hz, 1H), 5.10 (d, J=1.5 Hz, 1H), 4.32-4.28 (m, 2H), 4.15 (dd, J=11.4, 8.0 Hz, 1H), 4.08-4.03 (m, 3H), 4.01-3.93 (m, 4H), 3.85-3.78 (m, 5H), 3.77-3.59 (m, 5H), 3.48 (dd, J=10.1, 7.6 Hz, 1H), 3.30 (dd, J=9.3, 9.2 Hz, 1H), 2.33 (dd, J=8.3, 6.9 Hz, 2H), 2.24 (dd, J=7.5, 7.5 Hz, 2H), 1.65-1.55 (m, 4H), 1.29-1.26 (m, 48H), 0.91-0.87 (m, 6H). ³¹P NMR (121.5 MHz, CDCl₃) δ 4.63. HRMS-ESI [M−H]⁻ calcd for C₅₃H₉₉NO₂₂P: 1132.6396. Found 1132.6382.

### Example 4

### Synthesis of 2,6-(Di-O-α-D-mannopyranosyl)-1-O-(2-hexadecanoyl-1-O-hexadecyl-sn-glycero-3-phosphoryl)-D-myo-inositol (4.6)

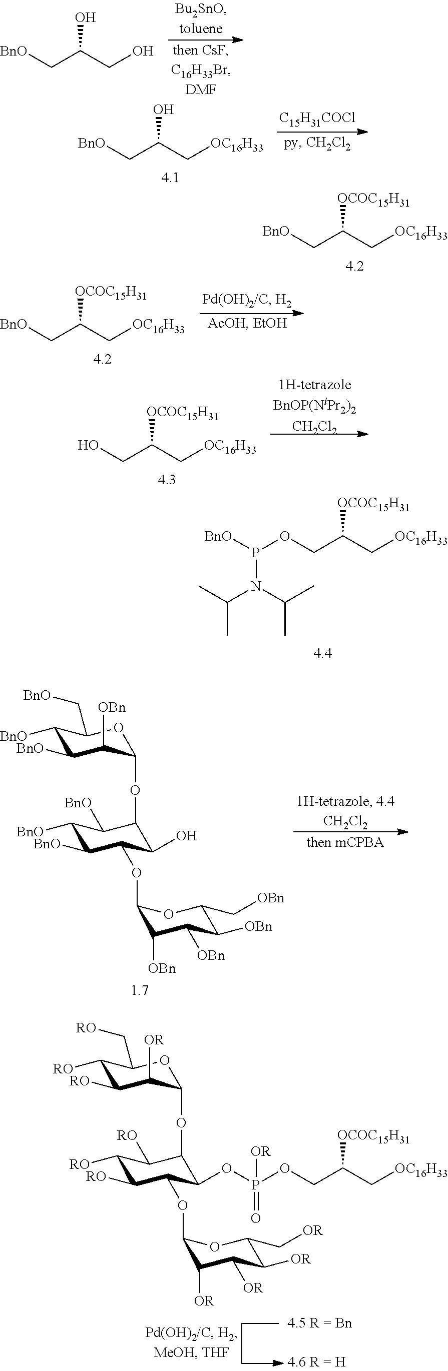

1-O-Hexadecyl-3-O-(4-methoxybenzyl)-sn-glycerol (4.1). A mixture of dibutyltin oxide (627 mg, 2.52 mmol) and 3-O-(4-methoxy-benzyl)sn-glycerol (534 mg, 2.52 mmol) in toluene was reluxed under argon for 3 h. After cooling the solvent was removed in vacuo and the residue dissolved in DMF (10 mL). CsF (410 mg, 2.70 mmol) and bromohexadecane (1.10 mL, 3.61 mmol) were added to the stirred solution that was heated to 80° C. for 16 h. The reaction mixture was quenched with H₂O (100 mL) and extracted with Et₂O (2×150 mL) and the ethereal extract washed with H₂O (100 mL) and dried (MgSO₄). After filtration, the solvent was removed in vacuo and the residue purified by column chromatography on silica gel. Elution with EtOAc/light petroleum (1:4 to 3:7) afforded the title compound 4.1 (789 mg, 1.81 mmol, 72%) as an oil. [α]_{D}²⁰=+1.0 (c 7.7, CHCl₃). [α]_{D}²⁰=+2.1 (c 0.70, CH₂Cl₂). ¹H NMR (300 MHz, CDCl₃) δ 7.30-7.22 (m, 2H), 6.90-6.84 (m, 2H), 4.48 (s, 2H), 3.98-3.93 (m, 1H), 3.79 (s, 3H), 3.52-3.40 (m, 6H), 2.49 (d, J=4.2 Hz, 1H), 1.60-1.49 (m, 2H), 1.38-1.20 (m, 26H), 0.92-0.85 (m, 3H). ¹³C NMR (75 MHz, CDCl₃) δ 159.4, 130.2, 129.4, 113.9, 73.2, 71.9, 71.8, 71.2, 69.6, 55.3, 32.0, 29.7, 29.6, 29.5, 29.4, 26.2, 22.7, 14.2. HRMS-ESI [M+Na] calcd for C₂₇H₄₈O₄Na: 459.3450. Found 459.3442.

2-O-Hexadecanoyl-1-O-hexadecyl-3-O-(4-methoxybenzyl)-sn-glycerol (4.2). Palmitoyl chloride (0.300 mL, 0.982 mmol) was added dropwise to a stirred solution of alcohol 4.1 (270 mg, 0.618 mmol) and pyridine (0.300 mL, 3.71 mmol) in CH₂Cl₂ (15 mL) cooled to 0° C. After being stirred for 17 h at rt, the reaction mixture was quenched with H₂O (100 mL). The mixture was extracted with Et₂O (2×150 mL) and the ethereal extract washed with a 0.5M HCl solution (100 mL), saturated NaHCO₃ solution (100 mL), and dried (MgSO₄). After filtration, the solvent was removed in vacuo and the residue purified by column chromatography on silica gel. Elution with EtOAc/light petroleum (0:1 to 1:9) afforded the title compound 4.2 (384 mg, 0.569 mmol, 92%) as an oil. [α]_{D}²⁰=+1.0 (c 7.7, CHCl₃). ¹H NMR (300 MHz, CDCl₃) δ ¹H NMR (300 MHz, CDCl₃) δ 7.27-7.21 (m, 2H), 6.90-6.83 (m, 2H), 5.16 (quintet, J=5.1 Hz, 1H), 4.49-4.43 (m, 2H), 3.79 (s, 3H), 3.60-3.55 (m, 4H), 3.44-3.35 (m, 2H), 2.32 (t, J=7.5 Hz, 2H), 1.64-1.49 (m, 4H), 1.40-1.20 (m, 50H), 0.92-0.83 (m, 6H). ¹³C NMR (75 MHz, CDCl₃) δ 173.4, 159.3, 130.2, 129.3, 113.8, 73.0, 71.7, 71.4, 69.3, 68.6, 55.3, 34.5, 32.0, 29.8, 29.6, 29.4, 29.2, 26.1, 25.1, 22.8, 14.2. HRMS-ESI [M+Na]⁺ calcd for C₄₃H₇₈O₅Na: 697.5747. Found 697.5734.

2-O-Hexadecanoyl-1-O-hexadecyl-sn-glycerol (4.3). A mixture of the benzyl ether 4.2 (380 mg, 0.563 mmol) and Pd(OH)₂/C (20%, 88 mg) in EtOH (15 mL) and AcOH (1.5 mL) was stirred under hydrogen for 4 h. The hydrogen was removed and the mixture filtered through Celite. The filtrate was concentrated in vacuo and the residue purified by column chromatography on silica gel. Elution with EtOAc/CH₂Cl₂ (1:50 to 1:20) afforded the title compound 4.3 (207 mg, 0.373 mmol, 66%) as an oil. [α]_{D}²⁰=−2.8 (c 0.72, CHCl₃). ¹H NMR (300 MHz, CDCl₃) δ 5.00 (quintet, J=5.2 Hz, 1H), 3.85-3.76 (m, 2H), 3.67-3.56 (m, 2H), 3.50-3.39 (m, 2H), 2.35 (d, J=7.4 Hz, 2H), 2.22 (t, J=6.2 Hz, 1H), 1.65-1.52 (m, 4H), 1.38-1.21 (m, 50H), 0.92-0.85 (m, 6H). ¹³C NMR (75 MHz, CDCl₃) δ 173.8, 72.9, 72.0, 70.1, 63.1, 34.5, 32.0, 29.8, 29.5, 29.4, 29.3, 292, 26.1, 25.1, 22.8, 14.2. HRMS-ESI [M+Na]⁺ calcd for C₃₅H₇₀O₄Na: 577.5172. Found 577.5165.

Benzyl (2-O-hexadecanoyl-1-O-hexadecyl-sn-glycero)-diisopropylphosphoramidite (4.4). 1H-Tetrazole (35 mg, 0.50 mmol) was added to a stirred solution of alcohol 4.3 (205 mg, 0.369 mmol) and benzyloxy-bis-(diisopropylamino)phosphine (262 mg, 0.775 mmol) in dry CH₂Cl₂ (20 mL). After stirring for 90 min at rt, the solvent was removed in vacua and the residue purified by column chromatography on silica gel. Elution with Et₃N/EtOAc/light petroleum (3:10:90) afforded the title compound 4.4 (267 mg, 0.337 mmol, 91%) as an oil. [α]_{D}²⁰=+4.9 (c 0.72, CHCl₃). ¹H NMR (300 MHz, CDCl₃) δ 7.37-7.23 (m, 5H), 5.13 (quintet, J=5.2 Hz, 1H), 4.74-4.62 (m, 2H), 3.83-3.35 (m, 8H), 2.30 (t, J=7.3 Hz, 2H), 1.65-1.48 (m, 4H), 1.32-1.16 (m, 62H), 0.90-0.83 (m, 6H). ¹³C NMR (75 MHz, CDCl₃) δ 173.3, 139.6, 128.3, 127.3, 127.0, 72.2, 71.7, 69.2, 65.5, 65.3, 62.3, 62.1, 61.9, 43.2, 43.1, 34.6, 32.0, 29.8, 29.7, 29.6, 29.4, 29.2, 26.2, 25.1, 24.8, 24.7, 24.6, 22.8, 14.2. ³¹P NMR (121.5 MHz, CDCl₃) δ 149.5, 149.2. HRMS-ESI, [M+H]⁺ calcd for C₄₈H₉₁NO₅P: 729.6635. Found 792.6638.

3,4,5-Tri-O-benzyl-2,6-di-O-(2,3,4,6-tetra-O-benzyl-α-D-mannopyranosyl)-1-O-(2-O-hexadecanoyl-1-O-hexadecyl-sn-glycero-3-benzylphosphoryl)-D-myo-inositol (4.5). 1H-Tetrazole (21 mg, 0.30 mmol) was added to a stirred solution of 3,4,5-tri-O-benzyl-2,6-di-O-(2,3,4,6-tetra-O-benzyl-α-D-mannopyranosyl)-D-myo-inositol (1.7) (90 mg, 0.060 mmol) and phosphoramidite 4.4 (135 mg, 0.170 mmol) in dry CH₂Cl₂ (7 mL) cooled to 0° C. under argon. After stirring at it for 2 h the reaction mixture was cooled to −40° C. and a solution of m-CPBA (55%, 90 mg, 0.29 mmol) in CH₂Cl₂ (10 mL) was transferred by cannula into the reaction mixture. After being stirred at it for 1 h the reaction was quenched by addition of a 10% Na₂SO₃ solution (50 mL) and the combined mixture extracted with Et₂O (100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9 to 3:7) followed by a second column and elution with EtOAc/CH₂Cl₂ (1:50 to 1:20) afforded the title compound 4.5 (68 mg, 0.031 mmol, 52%) as an oil. ¹H NMR (300 MHz, CDCl₃) δ 7.38-7.00 (m, 60H), 5.53-5.51 (m, 1H), 5.33-5.32 (m, 1H), 5.05-4.40 (m, 23H), 4.30-3.78 (m, 17H), 3.56-3.20 (m, 9H), 2.23-2.10 (m, 2H), 1.58-1.41 (m, 4H), 1.31-1.15 (m, 50H), 0.89-0.82 (m, 6H). ¹³C (75 MHz, CDCl₃) selected signals δ 173.3, 99.9, 98.9. ³¹P NMR (121.5 MHz, CDCl₃) δ 0.26, 0.00. HRMS-ESI [M+Na]⁺ calcd for C₁₃₇H₁₇₃O₂₂NaP: 2224.2054. Found 2224.2051.

2,6-(Di-O-α-D-mannopyranosyl)-1-O-(1-O-hexadecanoyl-2-O-hexadecyl-sn-glycero-3-phosphoryl)-D-myo-inositol (4.6). Pd(OH)₂/C (20%, 25 mg) was added to a stirred solution of the fully substituted 4.5 (68 mg, 0.031 mmol) in THF/MeOH (2:3, 5 mL). The mixture was stirred under hydrogen for 4 h at rt and the hydrogen was replaced with argon. The mixture was filtered through Celite and the filtrate concentrated in vacuo and the residue was purified by column chromatography on silica gel. Elution with H₂O/MeOH/CHCl₃ (0:4:7 to 0.2:4:7 to 0.4:4:7 to 0.6:4:7) afforded the title compound 4.6 (26 mg, 0.023 mmol, 74%) as a white powder. [α]_{D}²⁰=+39 (c 0.40, H₂O/CH₃OH/CHCl₃, 0.6:4:7). ¹H NMR (500 MHz, CDCl₃/CD₃OD/D₂O, 70:40:6) δ 5.28-5.20 (m, 1H), 5.15 (br s, 1H), 5.12 (br s, 1H), 4.33 (br s, 1H), 4.12-3.96 (m, 8H), 3.87-3.60 (m, xxH), 3.55-3.43(m, xxH), 3.30 (t, J=xx Hz, 1H), 2.41 (t, J=7.2 Hz, 2H), 1.65-1.55 (m, 4H), 1.33-1.22 (m, 50H), 0.86 (t, J=6.9 Hz, 6H). ¹³C (125 MHz, CDCl₃) δ 176.0, 103.0, 102.9, 79.9, 79.8, 78.2, 78.1, 74.8, 74.4, 74.4, 74.2, 73.5, 73.5, 73.0, 72.1, 72.1, 71.8, 71.6, 70.7, 68.4, 68.4, 65.4, 65.4, 62.8, 62.6, 35.7, 33.2, 31.0, 30.9, 30.7, 30.6, 30.4, 27.4, 26.4, 23.9, 15.0, 10.0. ³¹P NMR (121.5 MHz, CDCl₃/CD₃OD/D₂O, 70:40:6) δ 0.63. HRMS-ESI [M−H]⁻ calcd for C₅₃H₁₀₀O₂₂P: 1119.6444. Found 1119.6456.

### Example 5

### Synthesis of 6-(O-α-D-Mannopyranosyl)-1-O-(1-hexadecanoyl-2-O-hexadecyl-sn-glycero-3-phosphoryl)-D-myo-inositol (5.3)

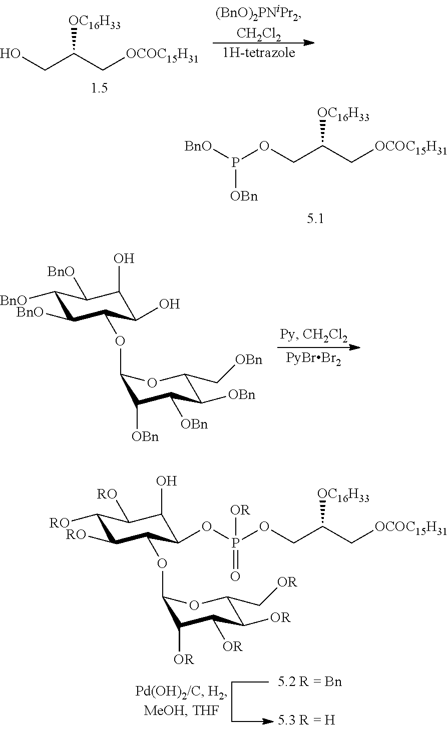

Dibenzyl (1-O-hexadecanoyl-2-O-hexadecyl-sn-glycero)-phosphite (5.1). 1H-Tetrazole (116 mg, 1.66 mmol) was added to a stirred solution of alcohol 1.5 (231 mg, 0.416 mmol) and bis-benzyloxy-diisopropylaminophosphine (200 μL, 0.608 mmol) in dry CH₂Cl₂ (20 mL). After stirring for 4 h at rt, the solvent was removed in vacuo and the residue purified by column chromatography on silica gel. Elution with Et₃N/EtOAc/light petroleum (1:5:95) afforded the title compound 5.1 (223 mg, 0.279 mmol, 67%) as an oil. [1:x12=+5.1 (c 2.5, CHCl₃). ¹H NMR (300 MHz, CDCl₃) δ 7.40-7.23 (m, 5H), 4.88 (d, J=9.0 Hz, 4H), 4.20 (dd, J=11.6, 4.4 Hz, 1H), 4.08 (dd, J=11.6, 5.6 Hz, 1H), 3.90-3.79 (m, 2H), 3.56 (quintet, J=4.9 Hz, 1H), 3.49 (t, J=6.7 Hz, 2H), 2.29 (t, J=7.7 Hz, 2H), 1.65-1.48 (m, 4H), 1.32-1.16 (m, 50H), 0.90-0.82 (m, 6H). ¹³C NMR (75 MHz, CDCl₃) δ 174.0, 138.6, 138.5, 128.8, 128.1, 127.9; 77.1, 71.0, 64.8, 64.7, 64.6, 63.5, 62.0, 61.9, 34.6, 32.3, 30.4, 29.9, 29.7, 29.7, 29.6, 26.4, 25.3, 23.1, 14.5. ³¹P NMR (121.5 MHz, CDCl₃) δ 140.5. HRMS-ESI, [M+H]⁺ calcd for C₄₉H₈₄O₆P: 799.6006. Found 799.6005.

3,4,5-Tri-O-benzyl-6-O-(2,3,4,6-tetra-O-benzyl-α-D-mannopyranosyl)-1-O-(1-O-hexadecanoyl-2-O-hexadecyl-sn-glycero-3-benzylphosphonositol (5.2). A solution of 3,4,5-tri-O-benzyl-6-O-(2,3,4,6-tetra-O-benzyl-α-D-mannopyranosyl)-D-myo-inositol (119 mg, 0.122 mmol) and phosphite 5.1 (166 mg, 0.208 mmol) was stirred in dry CH₂Cl₂ (8 mL) at rt for 45 min then cooled to −40° C. when pryidinium tribromide (90 mg, 0.28 mmol) was added. After 15 min the reaction was quenched by addition of a 10% Na₂S₂O₃ solution (50 mL) and the combined mixture extracted with Et₂O (100 mL). The ethereal extract was washed with a saturated NaHCO₃ solution (2×50 mL) and dried (MgSO₄). After filtration, the solvent was removed in vacuo and the residue purified by column chromatography on silica gel. Elution with EtOAc/light petroleum (1:9 to 1:4) afforded the title compound 5.2 (102 mg, 0.061 mmol, 51%) as an oil. ¹H NMR (300 MHz, CDCl₃) δ 7.38-7.00 (m, 40H), 5.43-5.40 (m, 1H), 5.10-3.82 (m, 28H), 3.59-3.22 (m, 6H), 2.81 (brs, 0.55H), 2.63 (brs, 0.45H), 2.29-2.19 (m, 2H), 1.57-1.39 (m, 4H), 1.31-1.15 (m, 50H), 0.89-0.82 (m, 6H). ³¹P NMR (121.5 MHz, CDCl₃) δ 0.00, −0.58. HRMS-ESI [M+Na]⁺ calcd for C₁₀₃H₁₃₉O₁₇NaP: 1701.9648. Found 1701.9667.

6-O-(α-D-Mannopyranosyl)-1-O-(1-O-hexadecanoyl-2-O-hexadecyl-sn-glycero-3-phosphoryl)-D-myo-inositol (5.3). Pd(OH)₂/C (20%, 15 mg) was added to a stirred solution of the fully substituted 5.2 (18 mg, 0.011 mmol) in THF/MeOH (2:3, 2.5 mL). The mixture was stirred under hydrogen for 3.5 h at rt and the hydrogen was replaced with argon. The mixture was filtered through Celite and the filtrate concentrated in vacuo and the residue was purified by column chromatography on silica gel. Elution with H₂O/MeOH/CHCl₃ (0:4:7 to 0.2:4:7 to 0.4:4:7 to 0.6:4:7) afforded the title compound 5.3 (8.0 mg, 0.0083 mmol, 78%) as a white powder. [α]_{D}²⁰=+31 (c 0.40, H₂O/CH₃OH/CHCl₃, 0.6f4:7). ¹H NMR (500 MHz, CDCl₃/CD₃OD/D₂O, 70:40:6) δ 5.13 (s, 1H), 4.31 (dd, J=11.8, 3.0 Hz, 1H), 4.13 (dd, J=11.9, 7.3 Hz, 1H), 4.10-4.08 (m, 1H), 4.07-4.02 (m, 1H), 3.99-3.91 (m, 3H), 3.87-3.79 (m, 3H), 3.77-3.71 (m, 2H), 3.69-3.62 (m, 4H), 3.56-3.51 (m, 1H), 3.42 (dd, J=9.9, 2.8 Hz, 1H), 3.29 (t, J=9.2 Hz, 1H), 2.35 (t, J=7.2 Hz, 2H), 1.65-1.53 (m, 4H), 1.36-1.24 (m, 50H), 0.89 (t, J=6.9 Hz, 6H). ¹³C (125 MHz, CDCl₃) δ 175.0, 101.8, 78.6, 77.2, 73.5, 73.1, 71.5, 71.4, 71.1, 70.9, 70.5, 67.3, 64.7, 64.4, 61.6, 34.6, 32.2, 30.0, 29.9, 29.8, 29.6, 29.5, 26.3, 25.2, 22.9, 14.2. ³¹P NMR (121.5 MHz, CDCl₃/CD₃OD/D₂O, 70:40:6) δ 4.3. HRMS-ESI [M+H]⁺ calcd for C₄₇H₉₀O₁₇P: 957.5916. Found 957.5945.

### Example 6

### Synthesis of 2-(O-α-D-mannopyranosyl)-1-O-(1-hexadecanoyl-2-O-hexadecyl-sn-glycero-3-phosphoryl)-D-myo-inositol (6.2)

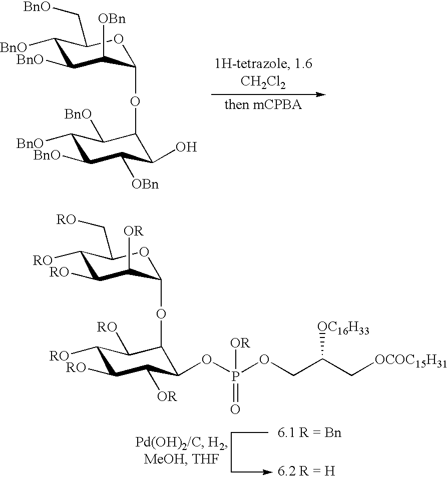

3,4,5,6-Tetra-O-benzyl-2-O-(2,3,4,6-tetra-O-benzyl-α-D-mannopyranosyl)-1-O-(1-O-Hexadecanoyl-2-O-hexadecyl-sn-glycero-3-benzylphosphoryl)-D-myo-inositol (6.1). A mixture of 3,4,5,6-tetra-O-benzyl-2-O-(2,3,4,6-tetra-O-benzyl-α-D-mannopyranosyl)-D-myo-inositol (40 mg, 0.038 mmol) and phosphoramidite 1.6 (76 mg, 0.090 mmol) were concentrated in vacuo from dry toluene. The residue was dissolved in dry CH₂Cl₂ (5 mL) and 1-H-tetrazole (7.9 mg, 0.11 mmol) was added at 0° C. under argon. After stirring at rt for 1 h the reaction mixture was cooled to −40° C. and a solution of m-CPBA (50%, 65 mg, 0.19 mmol) in dry CH₂Cl₂ (2 mL) was transferred by cannula into the reaction mixture. The reaction was warmed to it and after being stirred for 1.5 h the reaction was quenched by the addition of a 10% Na₂SO₃ solution (50 mL) and the combined mixture extracted with Et₂O (2×80 mL). The ethereal extract was washed with a saturated NaHCO₃ solution (3×50 mL), dried (MgSO₄) then filtered and the solvent removed. The crude residue was purified on silica gel with EtOAc/light petroleum (1:9) followed by a second purification on silica gel with EtOAc:toluene (1:5) to afford the title compound 6.1 (38 mg, 0.017 mmol, 32%) as an oil. ¹H NMR (300 MHz, CDCl₃) δ 7.43-7.15 (m, 45H), 5.37 (br s, 1H), 5.10-4.47 (m, 19H), 4.33-3.66 (m, 12H), 3.54-3.24 (m, 7H), 2.20 (t, J=7.6 Hz, 2H), 1.60-1.36 (m, 4H), 1.29-1.17 (m, 50H), 0.92-0.86 (m, 6H). ¹³C (75 MHz, CDCl₃) selected signals δ 173.3, 98.9. ³¹P NMR (121.5 MHz, CDCl₃) δ 0.54, 0.00. HRMS-ESI [M+H]⁺ calcd for C₁₁₀H₁₄₆O₁₇P: 1792.0117. Found 1792.0144.

2-O-(0-Benzyl-α-D-mannopyranosyl)-1-O-(1-O-Hexadecanoyl-2-O-hexadecyl-sn-glycero-3-benzylphosphoryl)-D-myo-inositol (6.2). Pd(OH)₂/C (20%, 17 mg) was added to a stirred solution of fully substituted 6.1 (20 mg, 0.011 mmol) in THF/MeOH (2:3, 2.5 mL). The mixture was stirred under hydrogen for 3.5 h at rt and the hydrogen was replaced with argon. The mixture was filtered through Celite and the filtrate concentrated in vacuo. The residue was lyophilized to afford 6.2 (8 mg, 0.0083 mmol, 74%) as a white powder. ¹H NMR (300 MHz, CDCl₃/CD₃OD/D₂O, 70:40:6) δ 5.14 (br s, 1H), 4.51-3.26 (m, 25H), 2.34 (t, J=7.7 Hz, 2H), 1.65-1.48 (m, 4H), 1.38-1.19 (m, 50H), 0.89 (t, J=6.6 Hz, 6H). ³¹P NMR (121.5 MHz, CDCl₃/CD₃OD/D₂O, 70:40:6) δ 0.8. HRMS-ESI [M+H]⁺ calcd for C₄₇H₉₁O₁₇NaP: 957.5892. Found 957.5897.

### Example 7

### Synthesis of 1,3-(Di-O-α-D-mannopyranosyl)-1-O-(1-O-hexadecanoyl-2-O-hexadecyl-sn-glycero-3-phosphoryl)-glycerol (7.10)

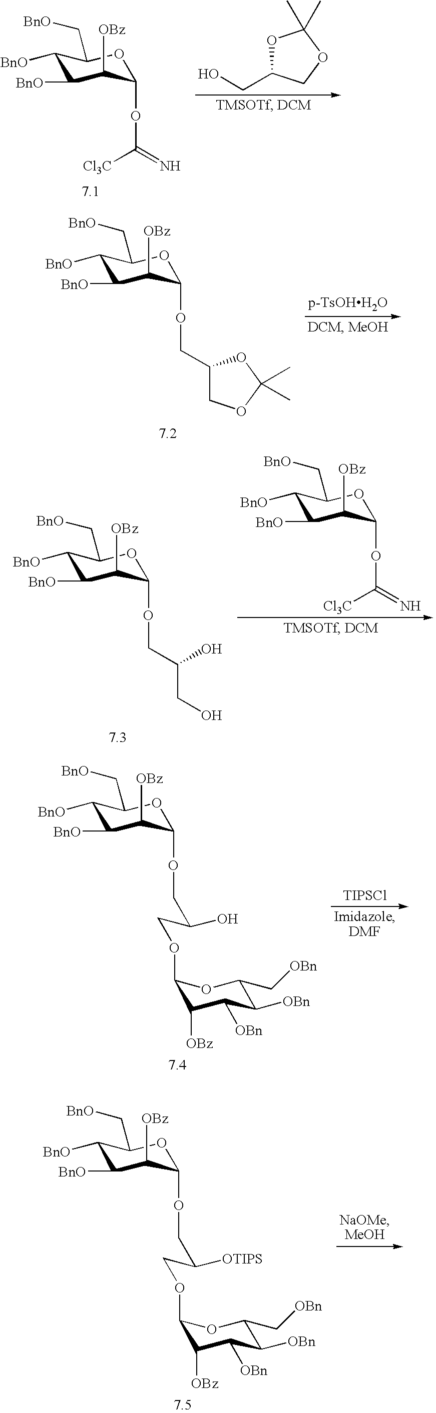

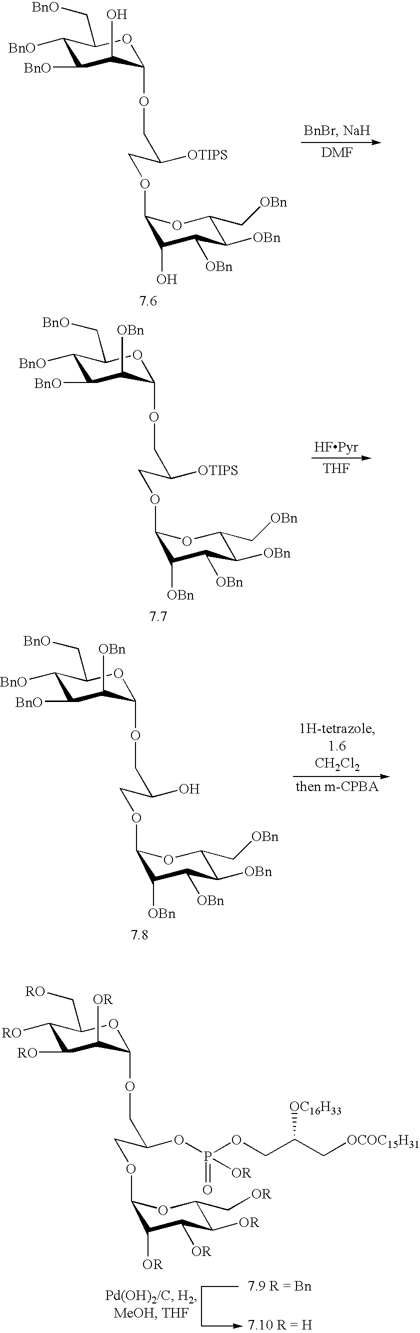

2-O-benzoyl-3,4,6-tri-O-benzyl-α-D-mannopyranosyl-1-O-(1,2-O-isopropylidene-sn-glycerol) (7.2). TMSOTf (26 μL, 0.143 mmol) was added dropwise to a stirred mixture of 2-O-benzoyl-,3,4,6-tri-O-benzyl-α-D-mannopyranosyl trichloroacetimidate 7.1 (500 mg, 0.715 mmol), 1,2-O-isopropylidene-sn-glycerol (133 μL, 1.073 mmol) and 4 Å molecular sieves (300 mg) in CH₂Cl₂ (10 ml) at −60° C. The reaction mixture was allowed to warm to 0° C. over 2 h when triethylamine (150 μl) was added. The mixture was then filtered through Celite and the filter cake washed with further CH₂Cl₂ (100 ml). The solvent mixture was removed and the residue purified by column chromatography on silica gel. Elution with EtOAc/light petroleum (1:8) afforded the title compound 7.2 (432 mg, 0.646 mmol, 90%) as an oil. [α]_{D}²⁰=−3.48 (c 2.30, CHCl₃). ¹H NMR (300 MHz, CDCl₃) δ 8.05 (d, J=7.2 Hz , 2H), 7.54 (t, J=7.2 Hz, 1H), 7.39-7.18 (m, 17H), 5.66 (br s, 1H), 5.00 (d, J=3.0 Hz, 1H), 4.86 (d, J=10.8 Hz, 1H), 4.78 (d, J=11.4 Hz, 1H), 4.70 (d, J=12.0 Hz, 1H), 4.58-4.52 (m, 3H), 4.33-4.20 (m, 1H), 4.13-4.02 (m, 3H), 3.90-3.82 (m, 2H), 3.78-3.65 (m, 3H), 3.55 (dd, J=5.9, 6.0 Hz, 1H), 1.39 (s, 3H), 1.36 (s, 3H). NMR (75 MHz, CDCl₃) δ 166.1, 138.8, 138.4, 133.5, 130.4, 128.8, 128.7, 128.4, 128.3, 128.0, 127.9, 110.1, 98.5, 78.5, 75.6, 74.8, 74.6, 73.8, 72.2, 72.0, 69.4, 69.3, 69.1, 67.0, 27.1, 25.8. HRMS-ESI [M+Na]⁺ calcd for C₄₀H₄₄O₉Na: 691.2883. Found 691.2886.

2-O-benzoyl-3,4,6-tri-O-benzyl-α-D-mannopyranosyl-1-O-sn-glycerol (7.3). 2-O-benzoyl-3,4,6-tri-O-benzyl-α-D-mannopyranosyl-1-O-(1,2-O-isopropylidene-Sn-glycerol) (7.2) (400 mg, 0.5981 mmol) was dissolved in a mixture of MeOH/DCM (ratio 1:1, 10 ml) and cooled 0° C. before the slow addition of p-tolunesulfonic acid monohydrate (57 mg, 0.299 mmol). After being stirred at rt over 2 h the reaction was quenched by addition of a saturated NaHCO₃ solution (50 mL) and the combined mixture extracted with CH₂Cl₂ (100 mL). The organic layer was washed with water (3×50 mL) and dried (MgSO₄). After filtration, the solvent was removed in vacuo and the residue purified by column chromatography on silica gel. Elution with EtOAc/light petroleum (1:8) afforded the title compound 7.3 (354 mg, 0.563 mmol, 94%) as an oil. [_{]}_{D}²⁰=−8.00 (c 1.0, CHCl₃). ¹H NMR (300 MHz, CDCl₃) δ 8.05 (d, J=7.2 Hz , 2H), 7.54 (t, J=7.2 Hz, 1H), 7.35-7.18 (m, 17H), 5.59 (br s, 1H), 4.97 (d, J=1.8 Hz, 1H), 4.86 (d, J=10.8 Hz, 1H), 4.76 (d, J=11.4 Hz, 1H), 4.69 (d, J=12.0 Hz, 1H), 4.65-4.42 (m, 3H), 4.09-3.96 (m, 2H), 3.93-3.84 (m, 2H), 3.79-3.72 (m, 3H), 3.65₇3.49 (m, 3H). ¹³C NMR (75 MHz, CDCl₃) δ 166.2, 138.5, 138.3, 133.6, 130.4, 128.8, 128.7, 128.4, 128.3, 128.0, 98.8, 78.5, 75.6, 74.7, 73.9, 72.3, 72.1, 70.9, 70.1, 69.6, 69.5, 63.9. HRMS-ESI [M+Na]⁺ calcd for C₃₇H₄₀O₉Na: 651.2570. Found 651.2573.

1,3-di-O-(2-O-benzoyl-3,4,6-tri-O-benzyl-α-D-mannopyranosyl)-1-O-sn-glycerol (7.4). TMSOTf (14 μL, 0.078 mmol) was added dropwise to a stirred mixture of 2-O-benzoyl-3,4,6-tri-O-benzyl-α-D-mannopyranosyl-1-O-sn-glycerol (7.3) (244 mg, 0.388 mmol), 2-O-benzoyl-,3,4,6-tri-O-benzyl-α-D-mannopyranosyl trichloroacetimidate (7.1) (271 mg, 0.388 mmol) and 4 Å molecular sieves (300 mg) in CH₂Cl₂ (7 ml) at −60° C. The reaction mixture was allowed to warm to 0° C. over 2 h when triethylamine (300 μL) was added. The mixture was filtered through Celite and the filter cake washed with further CH₂Cl₂ (100 ml). The solvent mixture was removed and the residue purified by column chromatography on silica gel. Elution with EtOAc/light petroleum (1:8) afforded the title compound 7.4 (265 mg, 0.227 mmol, 59%) as an oil. [α]_{D}²⁰=−3.33 (c 0.6, CHCl₃). ¹H NMR (300 MHz, CDCl₃) δ 8.05 (d, J=9.0 Hz , 4H), 7.54 (t, J=9.0 Hz, 2H), 7.38-7.17 (m, 34H), 5.62 (br s, 2H), 4.99 (d, J=1.8 Hz, 2H), 4.85 (d, J=10.8 Hz, 2H), 4.78 (d, J=11.4 Hz, 2H), 4.69 (d, J=12.0 Hz, 2H), 4.58-4.49 (m, 6H), 4.06-3.55 (m, 15H), 2.71 (br s, 1H). ¹³ C NMR (75 MHz, CDCl₃) δ 166.05, 138.7, 138.3, 133.5, 130.4, 128.7, 128.4, 128.0, 127.9, 98.9, 98.8, 78.5, 75.6, 74.6, 73.8, 72.4, 72.1, 70.9, 70.3, 70.1, 69.6, 69.4. HRMS-ESI [M+Na]⁺ calcd for C₇₁F1₇₂O₅Na: 1187.4769. Found 1187.4752.

1,3-di-O-(2-O-benzoyl-3,4,6-tri-O-benzyl-α-D-mannopyranosyl)-1-O-(2-triisopropyisilane)-sn-glycerol (7.5). Imidazole (35 mg, 0.515 mmol) was added to a solution of 1,3-di-O-(2-O-benzoyl-3,4,6-tri-O-benzyl-α-D-mannopyranosyl)-1-O-sn-glycerol (7.4) (200 mg, 0.172 mmol) in DMF (5 ml) cooled to 0° C. under argon. TIPSCI (73 μL, 0.343 mmol) was added and the reaction mixture was stirred at 40° C. for 3 h. The reaction mixture was diluted with diethyl ether (150 ml) and was washed with water and brine solution, dried (MgSO₄). After filtration, the solvent was removed in vacuo and the residue purified by column chromatography on silica gel. Elution with EtOAc/light petroleum (1:9) afforded the title compound 7.5 (192 mg, 0.145 mmol, 85%) as an oil. [α]_{D}²⁰=+4.50 (c 1.1, CHCl₃). ¹H NMR (300 MHz, CDCl₃) δ 8.05 (d, J=7.2 Hz , 4H), 7.54 (t, J=7.2 Hz, 2H), 7.37-7.17 (m, 34H), 5.64 (dt, J 6.6, 2.4 Hz, 2H), 4.98 (dd, J=1.8, 3.6 Hz, 2H), 4.86 (d, J=12.9 Hz, 2H), 4.79-4.69 (m, 4H), 4.59-4.45 (m, 6H), 4.16-4.05 (m, 5H), 3.91-3.67 (m, 8H), 3.62-3.43 (m, 2H), 1.04 (s, 21H). ¹³C NMR (75 MHz, CDCl₃) δ 165.9, 138.9, 138.4, 133.4, 130.4, 128.75, 128.7, 128.6, 128.3, 128.2, 127.9, 98.9, 98.7, 78.8, 75.5, 74.5, 73.8, 72.3, 72.0, 71.9, 70.8, 70.3, 70.1 69.4, 69.3, 69.2, 18.5, 18.1, 12.9. HRMS-ESI [M+Na]⁺ calcd for C₈₀H₉₂O₁₈NaSi: 1343.6103. Found 1343.6069.

1,3-di-O-(3,4,6-tri-O-benzyl-α-D-mannopyranosyl)-1-O-(2-triisopropylsilane)-sn-glycerol (7.6). 1,3-Di-O-(2-O-benzoyl-3,4,6-tri-O-benzyl-cc-D-mannopyranosyl)-1-O-(2-triisopropylsilane)-sn-glycerol (7.5) (185 mg, 0.140 mmol) was suspended in methanol (5 ml) before NaOMe solution (30% solution in methanol, 50 μL) was added and the reaction was stirred for 12 h at room temperature. Amberlite-120 H+ resin was added to neutralise the reaction. After filtration, the solvent was removed in vacuo and the residue purified by column chromatography on silica gel. Elution with EtOAc/light petroleum (1:9) afforded the title compound 7.6 (135 mg, 0.121 mmol, 87%) as an oil. [α]_{D}²⁰=+50.0 (c 1.0, CHCl₃). ¹H NMR (300 MHz, CDCl₃) δ 7.32-7.15 (m, 30H), 4.89 (br s, 2H), 4.83 (dd, J 10.8, 2.7 Hz, 2H), 4.69-6.2 (m, 6H), 4.52-4.45 (m, 4H), 4.03-3.99 (m, 3H), 3.90-3.93 (m, 4H), 3.75-3.64 (m, 8H), 3.45-3.37 (m; 2H), 2.40 (d, J 10.2 Hz, 2H), 1.04 (s, 21H). ¹³C NMR (75 MHz, CDCl₃) δ 138.8, 138.7, 138.3, 128.9, 128.7, 128.1, 127.9, 100.3, 99.9, 80.6, 75.4, 74.6, 73.8, 72.4, 71.7, 70.9, 70.2, 70.0, 69.3, 68.7, 18.5, 12.9. HRMS-ESI [M+Na]⁺ calcd for C₆₆H₉₄O₁₃NaSi: 1135.5579. Found 1135.5596.

1,3-Di-O-(2,3,4,6-tetra-O-benzyl-α-D-mannopyranosyl)-1-O-(2-triisopropylsilane)-sn-glycerol (7.7). Sodium hydride (60% dispersion in mineral oil, 9 mg, 0.364 mmol) was added to a stirred solution of 1,3-di-O-(3,4,6-tri-O-benzyl-α-D-mannopyranosyl)-1-O-(2-triisopropylsilane)-sn-glycerol (7.6) (135 mg, 0.121 mmol) in DMF (2 ml) cooled to 0° C. After 30 min Benzyl bromide (40 μL, 0.303 mmol) was added and the reaction mixture was stirred for a further 3 h when NH₄Cl solution (75 ml) and water (75 ml) were added. The mixture was extracted with diethyl ether (100 ml) and the combined ethereal extracts were washed with water and dried (MgSO₄). After filtration, the solvent was removed in vacuo and the residue purified by column chromatography on silica gel. Elution with EtOAc/light petroleum (1:9) afforded the title compound 7.7 (140 mg, 0.108 mmol, 89%) as an oil. [α]_{D}²⁰=+20.8 (c 1.2, CHCl₃). ¹H NMR (300 MHz, CDCl₃) δ 7.32-7.15 (m, 40H), 4.88-4.83 (m, 4H), 4.72-4.65 (m, 5H), 4.61-4.57 (m, 5H), 4.52-4.44 (m, 4H), 4.06-3.96 (m, 3H), 3.89-3.85 (m, 2H), 3.82-3.61 (m, 10H), 3.39-3.27 (m, 2H), 0.97 (s, 21H). ¹³C NMR (75 MHz, CDCl₃) δ 138.6, 138.5, 138.4, 138.3, 128.3, 128.25, 127.85, 127.75, 127.7, 127.6, 127.5, 98.6, 98.5, 80.5, 80.4, 74.9, 74.85, 74.8, 74.6, 73.4, 74.6, 72.2, 72.1, 70.7, 70.1, 69.5, 69.2, 18.1, 12.5. HRMS-ESI [M+Na]⁺ calcd for C₈₀H₉₆O₁₃NaSi: 1315.6518. Found 1315.6522.

1,3-di-O-(2,3,4,6-tetra-O-benzyl-α-D-mannopyranosyl)-1-O-sn-glycerol (7.8). A solution of HF pyr (40 M, 300 μL) was added to a stirred solution of 1,3-di-O-(2,3,4,6-tetra-O-benzyl-α-D-mannopyranosyl)-1-O-(2-triisopropylsilane)-sn-glycerol (7.7) (130 mg, 0.10 mmol) in THF (5 ml) cooled to 0° C. After 1 h, the mixture was diluted with EtOAc (100 ml) and washed with saturated aqueous solution of NaHCO₃ and dried (MgSO₄). After filtration, the solvent was removed in vacuo and the residue purified by column chromatography on silica gel. Elution with EtOAc/light petroleum (1:9) afforded the title compound 7.8 (110 mg, 0.097 mmol, 97%) as an oil. [α]_{D}²⁰=+35.6 (c 2.5, CHCl₃). ¹H NMR (300 MHz, CDCl₃) δ 7.37-7.16 (m, 40H), 4.86-4.82 (m, 4H), 4.76-4.66 (m, 4H), 4.64-4.57 (m, 6H), 4.51-4.47 (m, 4H), 3.98-3.83 (m, 5H), 3.78-3.69 (m, 8H), 3.67-3.41 (m, 4H), 2.77 (br s, 1H). ¹³C NMR (75 MHz, CDCl₃) δ 138.8, 138.7, 128.7, 128.4, 128.1, 127.9, 99.3, 99.1, 80.4, 75.4, 73.8, 73.1, 72.6, 70.3, 69.9, 69.8, 69.6. HRMS-ESI [M+Na]⁺ calcd for C₇₁H₇₆O₁₃Na: 1159.5184. Found 1159.5158.

1,3-Di-O-(2,3,4,6-tetra-O-benzyl-α-D-mannopyranosyl)-1-O-(1-O-hexadecanoyl-2-O-hexadecyl-sn-glycero-3-benzylphosphoryl)-glycerol (7.9). 1H-Tetrazole (13 mg, 0.185 mmol) was added to a stirred solution of 1,3-di-O-(2,3,4,6-tetra-O-benzyl-α-D-mannopyranosyl)-1-O-sn-glycerol (7.8) (70 mg, 0.0615 mmol) and phosphoramidite 1.6 (149 mg, 0.185 mmol) in dry CH₂Cl₂ (3 ml) cooled to 0° C. under argon. After stirring at rt for 2 h the reaction mixture was cooled to −40° C. and a solution of m-CPBA (50%, 64 mg, 0.185 mmol) in CH₂Cl₂ (10 ml) was transferred by cannula into the reaction mixture. After being stirred at rt over 2 h the reaction was quenched by addition of a 10% Na₂SO₃ solution (50 mL) and the combined mixture extracted with Et₂O (100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9) afforded the title compound 7.9 (60 mg, 0.033 mmol, 53%) as an oil. [α]_{D}²⁰=+21.8 (c 2.8, CHCl₃). ¹H NMR (300 MHz, CDCl₃) δ 7.34-7.14 (m, 45H), 5.03-4.99 (m, 2H), 4.94 (br s, 1H), 4.90-4.80 (m, 3H), 4.71-57 (m, 8H), 4.50-4.44 (m, 6H), 4.03-3.94 (m, 4H), 3.87-3.52 (m, 13H), 3.40-3.31 (m, 2H), 2.31-2.04 (m, 2H), 1.62-1.59 (m 4H), 1.53-143 (m, 2H), 1.31-1.08 (m, 48H), 0.87 (t, J=6.6 Hz, 6H). ¹³C (75 MHz, CDCl₃) δ 173.4, 138.4, 128.6, 128.3, 127.9, 127.7, 127.5, 98.6, 98.0, 80.2, 75.8, 75.0, 74.7, 73.4, 72.7, 72.3, 72.05, 70.6, 69.2, 66.7, 66.3, 62.55, 34.9, 29.9, 29.7, 29.5, 29.4, 29.2, 26.0, 24.9, 22.7, 14.1. ³¹P NMR (121.5 MHz, CDCl₃) α 0.00, −0.09. HRMS-ESI [M+Na]⁺ calcd for C₁₁₃H₁₅₁O₁₉NaP: 1866.0485. Found 1866.0515.

1,3-(Di-O-α-D-mannopyranosyl)-1-O-(1-O-hexadecanoyl-2-O-hexadecyl-sn-glycero-3-phosphoryl)-glycerol (7.10). Pd(OH)₂/C (20%, 40 mg) was added to a stirred solution of the fully substituted 7.9 (60 mg, 0.0325 mmol) in THF/MeOH (2:3, 5 mL). The mixture was stirred under hydrogen for 2.5 h at rt and the hydrogen was replaced with argon. The mixture was filtered through Celite and the filtrate concentrated in vacuo. The residue was purified by column chromatography on silica gel. Elution with CHCl₃/CH₃OH/H₂O, (70:40:6) afforded the title compound 7.10, which was then lyophilized to afford 7.10 (28 mg, 0.027 mmol, 85%) as a white powder. [α]²⁰=+38.6 (c 1.4, CHCl₃/CH₃OH/H₂O, 70:40:6). ¹H NMR (300 MHz, CDCl₃/CD₃OD/D₂O, 70:40:6) δ 4.83 (dd, J=8.4, 1.2 Hz, 2H), 4.64-3.33 (m, 19H), 2.35 (t, J=7.5 Hz, 2H), 1.65-1.54 (m, 4H), 1.27-1.17 (m, 50H), 0.87 (t, J=6.9 Hz, 6H). ¹³C (75 MHz, CDCl₃) δ 175.1, 100.7, 100.6, 77.1, 73.4, 71.4, 71.2, 70.8, 67.7, 67.1, 66.9, 64.8, 64.5, 61.8, 34.6, 32.2, 30.2, 30.0, 69.7, 29.55, 26.4, 25.3, 22.9, 14.2. ³¹P NMR (121.5 MHz, CDCl₃/CD₃OD/D₂O, 70:40:6) δ 0.00. HRMS-ESI [M−H]⁻ calcd for C₃₀O₃₆O₁₉P: 1031.6283. Found 1031.6288.

### Example 8

### Synthesis of 6-(0-α-D-Mannopyranosyl)-2-(Oβ-D-mannopyranosyl-1-O-(1-hexadecanoyl-2-O-hexadecyl-sn-glycero-3-phosphoryl)-D-myo-inositol (8.6)

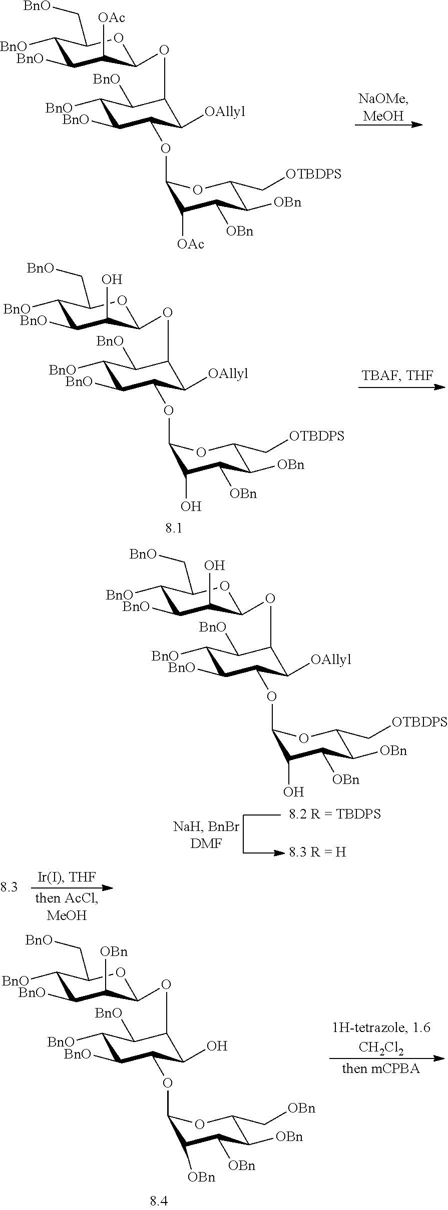

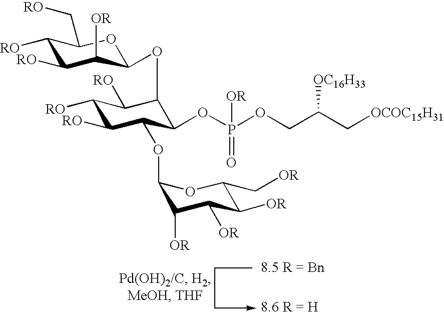

1-O-Allyl-2-O-(3,4,6-tri-O-benzyl-P-D-mannopyranosyl)-6-O-(3,4-di-O-benzyl-6-O-tert-butyldiphenylsilyl-α-D-mannopyranosyl)-3,4,5-tri-O-benzyl-D-myo-inositol (8.1). Sodium methoxide (30% solution in MeOH, 0.05 mL) was added drop-wise to a stirred solution of 1-O-allyl-2-O-(2-O-acetyl-3,4,6-tri-O-benzyl-(3-D-mannopyranosyl)-6-O-(2-O-acetyl-3,4-di-O-benzyl-6-O-tert-butyldiphenylsilyl-α-D-mannopyranosyl)-3,4,5-tri-O-benzyl-D-myo-inositol (82 mg, 0.052 mmol) in CH₂Cl₂:MeOH (3:5, 8 mL). After being stirred for 24 h the reaction mixture was diluted with aq NH₄Cl (sat., 50 mL). The aqueous phase was extracted with Et₂O (3×40 mL) and the combined organic extracts were washed with H₂O (100 ml). After drying (MgSO₄) and filtration the solvent was removed in vacuo and the residue purified by column chromatography on silica gel. Elution with EtOAc/light petroleum (1:4 to 2:3) afforded title compound 8.1 (52 mg, 0.035 mmol, 67%) as an oil. [α]_{D}²⁰=+4.5 (c 1.2, CHCl₃). ¹H NMR (300 MHz, CDCl₃) δ 7.72-7.59 (m, 4H), 7.40-6.88 (m, 46H), 5.96-5.82 (m, 1H), 5.43 (s, 1H), 5.23-5.10 (m, 2H), 4.92-4.43 (m, 18H), 4.24-3.20 (m, 19H), 2.88 (brs, 1H), 2.21 (brs, 1H), 1.01 (s, 9H). ¹³C NMR (75 MHz, CDCl₃) selected signals δ 100.2, 98.6, 27.0, 19.3. HRMS-ESI [M+Na]⁺ calcd for C₉₃H₁₃₂O₁₆SiNa: 1525.6835. Found 1525.6787.

1-O-Allyl-2-O-(3,4,6-tri-O-benzyl-β-D-mannopyranosyl)-6-O-(3,4-di-O-benzyl-6-O-tert-butyldiphenylsilyl-α-D-mannopyranosyl)-3,4,5-tri-O-benzyl-D-myo-inositol (8.2). Tetrabutylammonium fluoride (1M in THF, 4.00 mL, 4.00 mmol) was added to the silyl ether 8.1 (52 mg, 0.035 mmol) and stirred for 20 h. The solvent was removed in vacuo and the residue purified by column chromatography on silica gel. Elution with EtOAc/light petroleum (3:7 to 1:1 to 3:1) afforded the title compound 8.2 (38 mg, 0.030 mmol, 86%) as an oil. [α]_{D}²⁰=+6.6 (c 0.70, CH₂Cl₂). ¹H NMR (300 MHz, CDCl₃) δ 7.40-7.10 (m, 40H), 5.96-5.80 (m, 1H), 5.42 (s, 1H), 5.22-5.09 (m, 2H), 4.90-4.43 (m, 18H), 4.24-3.61 (m, 12H), 3.43-3.21 (m, 7H), 2.98 (brs, 1H), 2.41 (brs, 1H), 2.02 (brs, 1H). ¹³C NMR (75 MHz, CDCl₃) selected signals δ 117.7, 99.9, 98.8. HRMS-ESI [M+Na]⁺ calcd for C₇₇H₈₄O₁₆Na: 1287.5657. Found 1287.5679.

1-O-Allyl-2-O-(2,3,4,6-tetra-O-benzyl-(3-D-mannopyranosyl)-6-O-(2,3,4,6-tetra-O-benzyl-α-D-mannopyranosyl)-3,4,5-tri-O-benzyl-D-myo-inositol (8.3). Sodium hydride (60% dispersion in mineral oil, 19 mg, 0.48 mmol) was added to a stirred solution of triol 8.2 (38 mg, 0.030 mmol) in DMF (5 mL) cooled to 0° C. After 20 min benzyl bromide (30 μL, 0.25 mmol) was added and the reaction mixture stirred at it for 14 h when aq NH₄Cl (sat., 50 mL) was added. The mixture was extracted with ether (2×50 mL) and the combined ethereal extracts were washed with H₂O(2×50 mL). After drying (MgSO₄) and filtration the solvent was removed in vacua and the residue purified by column chromatography on silica gel. Elution with EtOAc/light petroleum (1:9 to 1:4 to 1:3) afforded the title compound 8.3 as an oil. [α]_{D}²⁰=−17 (c 0.74, CH₂Cl₂). ¹H NMR (300 MHz, CDCl₃) δ 7.50-7.45 (m, 2H), 7.40-7.15 (m, 53H), 5.88-5.70 (m, 1H), 5.62 (s, 1H), 5.18-4.40 (m, 25H), 4.22-4.09 (m, 4H), 3.97-3.70 (m, 9H), 3.42-3.19 (m, 7H). ¹³C NMR (75 MHz, CDCl₃) selected signals δ 117.5, 101.1, 98.2. HRMS-ESI [M+Na]⁺ calcd for C₉₈H₁₀₂O₁₆Na: 1557.7066. Found 1557.7092.

2-O-(2,3,4,6-Tetra-O-benzyl-β-D-mannopyranosyl)-6-O-(2,3,4,6-tetra-O-benzyl-α-D-mannopyranosyl)-3,4,5-tri-O-benzyl-D-myo-inositol (8.4). (1,5-Cyclooctadiene)bis(methyl-diphenylphosphine)iridium(I) hexafluorophosphate (2 mg, 0.002 mmol) was added to a stirred solution of allyl ether 8.3 (35 mg, 0.023 mmol) in THF (5 mL) under argon. This atmosphere was replaced with hydrogen for ca. 1 min and then, in turn, the hydrogen was replaced with argon. The mixture was stirred at 20° C. for 70 min, the solvent was removed in vacua and the residue dissolved with stirring in CH₂Cl₂/MeOH (1:1, 6 mL). Acetyl chloride (100 μL, 0.26 mmol) was added to this solution and stirring was continued for 3 h when solid NaHCO₃ (200 mg, 2.38 mmol) was added. The mixture was stirred for an additional 5 min when H₂O (50 mL) was added. This mixture was extracted with CHCl₃ (2×60 mL), and after drying (MgSO₄) and filtration the solvent was removed in vacuo and the residue purified by column chromatography on silica gel. Elution with EtOAc/light petroleum (1:4 to 3:7) afforded the title compound 8.4 as an oil (28 mg, 0.019 mmol, 83%). [α]_{D}²⁰=+8.2 (c 0.56, CH₂Cl₂). ¹H NMR (300 MHz, CDCl₃) δ 7.52-7.48 (m, 2H), 7.38-7.02 (m, 53H), 5.78 (s, 1H), 4.92-4.36 (m, 22H), 4.23-3.60 (m, 13H), 3.49-3.22 (m, 7H), 4.12-3.78 (m, 17H), 3.70-3.51 (m, 8H), 3.39-3.31 (m, 3H), 3.23 (t, J=9.5 Hz, 1H). ¹³C NMR (75 MHz, CDCl₃) selected signals δ 102.4, 97.6. Gated decoupled ¹³C NMR (75 MHz, CDCl₃) selected data, δ 102.4, 155 Hz, 6 97.6, ¹J_{C1′-H1′} 173 Hz. HRMS-ESI [M+Na]⁺ calcd for C₉₅H₉₃O₁₆Na: 1517.6753. Found 1517.6698.

3,4,5-Tri-O-benzyl-6-O-(2,3,4,6-tetra-O-benzyl-α-D-mannopyranosyl)-2-O-(2,3,4,6-tetra-O-benzyl-(3-D-mannopyranosyl)-1-O-(1-O-hexadecanoyl-2-O-hexadecyl-sn-glycero-3-benzylphosphoryl)-D-myo-inositol (8.5). 1H-Tetrazole (10 mg, 0.14 mmol) was added to a stirred solution of fully substituted 8.4 (94 mg, 0.063 mmol) and phosphoramidite 1.6 (80 mg, 0.101 mmol) in dry CH₂Cl₂ (8 mL) cooled to 0° C. under argon. After stirring at it for 2 h the reaction mixture was cooled to −40° C. and a solution of m-CPBA (55%, 65 mg, 0.207 mmol) in CH₂Cl₂ (10 mL) was transferred by cannula into the reaction mixture. After being stirred at it for 1 h the reaction was quenched by addition of a 10% Na₂SO₃ solution (50 mL) and the combined ixture extracted with Et₂O (2×100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9 to 3:17 to 1:4) followed by a second column and elution with acetone/toluene (1:50 to 1:25) afforded the title compound 8.5 (48 mg, 0.022 mmol, 35%) as an oil. ¹H NMR (300 MHz, CDCl₃) δ 7.52-7.43 (m, 2H), 7.30-7.00 (m, 58H), 5.53-5.49 (m, 1H), 5.15-3.21 (m, 50H), 2.25-2.18 (m, 2H), 1.63-1:38 (m, 41-I), 1.38-1.12 (m, 50H), 0.89-0.82 (m, 6H). ¹³C (75 MHz, CDCl₃) selected signals δ 173.7, 100.7, 98.4. ³¹P NMR (121.5 MHz, CDCl₃) δ 0.00, −0.11. HRMS-ESI [M+Na]^{{} calcd for C₁₃₇H₁₇₃O₂₂NaP: 2224.2053. Found 2224.2048.

6-(0-α-D-Mannopyranosyl)-2-(0-(3-D-mannopyranosyl)-1-O-(1-O-hexadecanoyl-2-O-hexadecyl-sn-glycero-3-phosphoryl)-D-myo-inositol (8.6). Pd(OH)₂/C (20%, 40 mg) was added to a stirred solution of the fully substituted 8.5 (45 mg, 0.020 mmol) in THF/MeOH (2:3, 5 mL). The mixture was stirred under hydrogen for 3.5 h at it and the hydrogen was replaced with argon. The mixture was filtered through Celite and the filtrate concentrated in vacuo and the residue was purified by column chromatography on silica gel. Elution with H₂O/MeOH/CHCl₃ (0:2:7 to 0:4:7 to 0.2:4:7 to 0.8:4:7) afforded the title compound 8.6 (9.0 mg, 0.0080 mmol, 40%) as a white powder. [α]_{D}²⁰ =+9.5 (c 0.40, H₂O/CH₃OH/CHCl₃, 0.6:4:7). ¹H NMR (300 MHz, D₂O/CD₃OD/CDCl₃, 0.5:4:7) δ 5.18 (br s, 1H), 4.82 (br s, 1H), 4.45-3.48 (m, 23H, 3.30-3.20 (m, 2H), 2.35 (t, J=7.5 H, 2H), 1.65-1.50 (m, 4H), 1.37-1.22 (m, 50H), 0.89 (ap t, J=6.5 Hz, 6H). ¹³C (75 MHz, CDCl₃) δ 175.2, 101.1(6), 101.1(5), 73.8, 73.2, 71.5, 70.9, 70.5, 67.4, 64.8, 64.4, 61.6, 34.6, 32.2, 30.0, 29.6, 26.3, 25.2, 22.9, 14.2. ³¹P NMR (121.5 MHz, D₂O/CD₃OD/CDCl₃, 0.5:4:7) δ 0.75 (s). HRMS-ESI (M−H)⁻ calcd for C₅₃H₁₀₀O₂₂P: 1129.6627. Found: 1119.6460.

### Example 9

### Synthesis of 2,6-(Di-O-α-D-mannopyranosyl)-1-O-(1-hexadecanoyl-2-O-hexadecyl-sn-glycero-3-carbonyloxy)-D-myo-inositol (9.2)

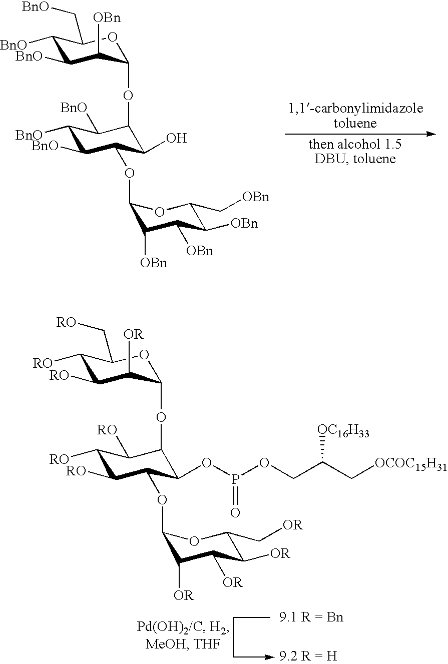

3,4,6-Tri-O-benzyl-2,6-di-O-(2,3,4,6-tetra-O-benzyl-α-D-mannopyranosyl)-1-O-(1-O-Hexadecanoyl-2-O-hexadecyl-sn-glycero-3-carbonyloxy)-D-myo-inositol (9.1). A solution of 1.6 (65 mg, 0.045 mmol) and 1,1′-carbonylimidazole (25 mg, 0.150 mmol) were concentrated in vacuo from dry toluene. The resulting residue was dissolved in dry toluene (4 mL) and heated at relfux, under argon, for 2 h. The reaction mixture was allowed to cool to rt, concentrated in vacuo and the residue diluted with chloroform (10 mL). The chloroform layer was washed with water (2×10 mL), dried (MgSO₄), filtered and concentrated in vacuo. A solution of the carbonylimidazole derivatised 1.6 (69 mg, 0.043 mmol) and alcohol 1.5 was concentrated in vacuo from dry toluene. The resulting residue was dissolved in dry toluene (2 mL) and 1,8-diazabicyclo[5.4.0]undec-7-ene (0.010 mL, 0.010 mmol) was added and the solution refluxed for 5 h. After cooling to rt the reaction mixture was concentrated in vacuo and purified on silica gel with EtOAc/light petroleum (1:10 to 1:6). A second purification on silica gel with EtOAc:toluene (1:8) afforded the title compound 9.1 (58 mg, 0.028 mmol, 64%) as an oil. [α]_{D}²⁰=+12 (c 0.01, CHCl₃). ¹H NMR (300 MHz, CDCl₃) δ 7.40-7.05 (m, 56H), 5.39-5.37 (m, 1H), 5.18-5.16 (m, 1H), 4.91-4.42 (m, 20H), 4.29 (d, J=13.0 Hz, 1H), 4.20-4.03 (m, 8H), 3.95-3.78 (m, 6H), 3.50-3.23 (m, 8H), 2.15 (td, J=7.4, 2.2 Hz, 2H), 1.59-1.47 (m, 2H), 1.40-1.09 (m, 50H), 0.90-0.86 (m, 6H). ¹³C NMR (75 MHz, CDCl₃) selected signals δ 173.2, 154.3, 99.0, 98.4. HRMS-ESI [M+Na]⁺ calcd for C₁₃₁H₁₆₆O₂₁Na: 2098.1819. Found 2098.1851.

3,4,5-Tri-O-benzyl-2,6-di-O-(α-D-mannopyranosyl)-1-O-(1-O-Hexadecanoyl-2-O-hexadecyl-sn-glypero-3-carbonyloxy)-D-myo-inositol (9.2). Pd(OH)₂/C (20%, 40 mg) was added to a stirred solution of the fully substituted 9.1 (20 mg, 0.010 mmol) in THF/MeOH (3:1, 4 mL). The mixture was stirred under hydrogen for 6 h at rt, after which time, the hydrogen was replaced with argon. The mixture was filtered through Celite and the filtrate concentrated in vacuo. The residue was lyophilized to afford 9.2 (10 mg, 0.009 mmol, 96%) as a white powder. [α]_{D}²⁰=+17 (c 0.004, CHCl₃). ¹H NMR (300 MHz, CDCl₃) δ 4.96-4.92 (m, 2H), 4.70 (dd, J=10.2, 1.9 Hz, 1H), 4.44-4.11 (m, 5H), 4.04-3.46 (m, 17H), 2.36 (t, J=7.6 Hz, 2H), 1.69-1.52 (m, 4H), 1.39-1.18 (m, 50H), 0.89 (m, 6H). ¹³C NMR (75 MHz, CDCl₃) selected signals δ 176.1, 156.2, 103.5, 103.1. HRMS-ESI [M+H]⁺ calcd for C₅₄H₁₀₀O₂₁Na: 1107.6655. Found 1107.6658.

### Example 10

### Synthesis of 2-(6-O-Hexadecanoyl-O-α-D-mannopyranosyl)-6-(0-α-D-mannopyranosyl)-1-O-(1-hexadecanoyl-2-O-hexadecyl-sn-glycero-3-phosphoryl)-D-myo-inositol (10.3)

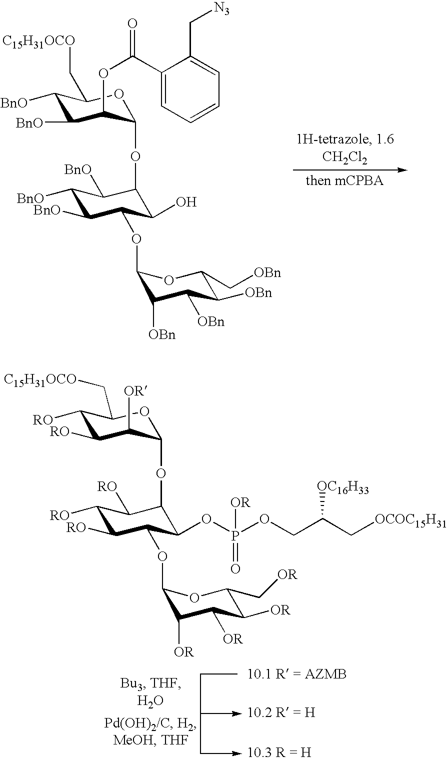

3,4,5-Tri-O-benzyl-2-O-(2-O-(2-azidomethylbenzyl)-3,4-di-O-benzyl-6-O-hexadecanoyl-α-D-mannopyranosyl)-6-O-(2,3,4,6-tetra-O-benzyl-α-D-mannopyranosyl)-1-O-(1-O-Hexadecanoyl-2-O-hexadecyl-sn-glycero-3-benzylphosphoryl)-D-myo-inositol (10.1). 1H-Tetrazole (13 mg, 0.19 mmol) was added to a stirred solution of 3,4,5-tri-O-benzyl-2-O-(2,3,4-tri-O-benzyl-6-O-hexadecanoyl-α-D-mannopyranosyl)-6-O-(2,3,4,6-tetra-O-benzyl-α-D-mannopyranosyl)-D-myo-inositol (106 mg, 0.062 mmol) and phosphoramidite 1.6 (147 mg, 0.186 mmol) in dry CH₂Cl₂ (8 mL) cooled to 0° C. under argon. After stirring at it for 3 h the reaction mixture was cooled to −40° C. and a solution of m-CPBA (50%, 86 mg, 0.25 mmol) in CH₂Cl₂ (10 mL) was transferred by cannula into the reaction mixture. After being stirred at it for 1 h the reaction was quenched by addition of a 10% Na₂SO₃ solution (50 mL) and the combined mixture extracted with Et₂O (100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9 to 1:4) followed by a second column and elution with acetone/toluene (1:50 to 3:97) afforded the title compound 10.1 (59 mg, 0.024 mmol, 39%) as an oil. ¹H NMR (300 MHz, CDCl₃) δ 8.04-7.98 (m, 1H), 7.58-7.43 (m, 2H), 7.36-7.00 (m, 51H), 5.68-5.50 (m, 2H), 5.33-5.27 (m, 1H), 5.10-5.02 (m, 2H), 4.95-4.39 (m, 20H), 4.36-3.80′ (m, 17H), 3.60-3.22 (m, 7H), 2.21-2.12 (m, 2H), 1.60-1.40 (m, 6H), 1.39-1.11 (m, 54H), 0.91-0.82 (m, 9H). ¹³C (75 MHz, CDCl₃) selected signals δ 173.6, 100.0, 99.0. ³¹P NMR (121.5 MHz, CDCl₃) δ 0.09, 0.00. HRMS-ESI [M+Na]⁺ calcd for C₁₄₇H₁₉₆N₃O₂₄NaP: 2441.3845. Found 2441.3855.

3,4,5-Tri-O-benzyl-2-O-(3,4-di-O-benzyl-6-O-hexadecanoyl-α-D-mannopyranosyl)-6-O-(2,3,4,6-tetra-O-benzyl-α-D-mannopyranosyl)-1-O-(1-O-Hexadecanoyl-2-O-hexadecyl-sn-glycero-3-benzylphosphoryl)-D-myo-inositol (10.2). A solution of the azidomethybenzyl ether 102 (58 mg, 0.024 mmol) in THF:H₂O (9:1, 10 mL) was degassed by evacuation and argon purging (process repeated 3 times) when tributyl phosphine (34 μL, 0.14 mmol) was added. After stirring at RT for 3 h toluene (20 mL) was added and the solvent removed in vacua The residue was purified by column chromatography on silica gel. Elution with EtOAc/light petroleum (1:4 to 3:7) afforded the title compound 10.2 (42 mg, 0.019 mmol, 78%) as an oil. ¹H NMR (300 MHz, CDCl₃) δ 7.40-6.99 (m, 50H), 5.44-5.40 (m, 2H), 5.17-4.39 (m, 21H), 4.18-3.78 (m, 18H), 3.58-3.20 (m, 7H), 2.55-2.48 (m, 2H), 2.26-2.15 (m, 4H), 1.60-1.39 (m, 6H), 1.37-1.12 (m, 74H), 0.90-0.80° (m, 9H). ¹³C (75 MHz, CDCl₃) selected signals δ 173.9, 101.9, 98.8. ³¹P NMR (121.5 MHz, CDCl₃) δ 0.00, −0.20. HRMS-ESI [M+Na]⁺ calcd for C₁₃₉H₁₉₁O₂₃NaP: 2282.3412. Found 2282.3401.

2-(6-O-Hexadecanoyl-O-α-D-mannopyranosyl)-6-(0-α-D-mannopyranosyl)-1-O-(1-hexadecanoyl-2-O-hexadecyl-sn-glycero-3-phosphoryl)-D-myo-inositol (10.3). Pd(OH)₂/C (20%, 36 mg) was added to a stirred solution of the fully substituted 10.2 (42 mg, 0.019 mmol) in THF/MeOH (2:3, 5 mL). The mixture was stirred under hydrogen for 3.5 h at it and the hydrogen was replaced with argon. The mixture was filtered through Celite and the filtrate concentrated in vacuo. The residue was purified by column chromatography on silica gel. Elution with H₂O/MeOH/CHCl₃ (0:2:7 to 0:4:7 to 0.2:4:7 to 0.8:4:7) afforded the title compound 10.3 (19 mg, 0.014 mmol, 75%) as a white powder. [α]_{D}²⁰=+30 (c 0.10, CHCl₃/CH₃OH/H₂O, 70:40:6). ¹H NMR (300 MHz, CDCl₃/CD₃OD/D₂O, 70:40:6) δ 5.13 (br s, 1H), 5.10 (br s, 1H), 4.38-4.23 (m, 2H), 4.20-3.40 (m, 24H), 3.27 (t, J=9.5 Hz, 1H), 2.37 (ap q, J=7.2 Hz, 4H), 1.65-1.50 (m, 6H), 1.38-1.20 (m, 74H), 0.89 (ap t, J=6.9 Hz, 9H).¹³C (75 MHz, CDCl₃) α 176.2, 176.2, 103.3, 80.2, 74.5, 72.5, 72.4, 72.0, 68.9, 36.0, 35.7, 33.6, 31.4, 31.0, 27.8, 26.7, 24.4, 15.6. ³¹P NMR (121.5 MHz, CDCl₃/CD₃OD/D₂O, 70:40:6) δ 0.84. HRMS-ESI [M−H]⁻ calcd for C₆₉H₁₃₀O₂₃P: 1357.8741. Found 1357.8729.

### Example 11

### Synthesis of α-D-galactopyranosyl-1-O-(1-O-Hexadecanoyl-2-O-hexadecyl-sn-glycero-3-phosphoryl)-glycerol (11.3)

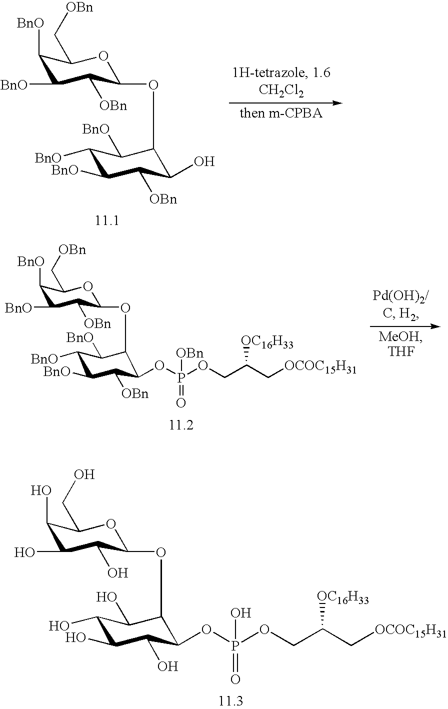

(2,3,4,6-tetra-O-benzyl-α-D-galactopyranosyl)-1-O-(1-O-Hexadecanoyl-2-O-hexadecyl-sn-glycero-3-benzylphosphoryl)-glycerol (11.2). 1H-Tetrazole (15 mg, 0.21 mmol) was added to a stirred solution of 2,3,4,6-tetra-O-benzyl-α-D-galactopyranosyl trichloroacetimidate (11.1) (112 mg, 0.1054 mmol) and phosphoramidite 1.6 (124 mg, 0.1615 mmol) in dry CH₂Cl₂ (3 mL) cooled to 0° C. under argon. After stirring at rt for 2 h the reaction mixture was cooled to −40° C. and a solution of m-CPBA (50%, 73 mg, 0.21 mmol) in CH₂Cl₂ (10 mL) was transferred by cannula into the reaction mixture. After being stirred at it over 2 h the reaction was quenched by addition of a 10% Na₂SO₃ solution (50 mL) and the combined mixture extracted with Et₂O (100 mL). The ethereal extract was washed with a saturated NaHCO₃ solution (3×50 mL) and dried (MgSO₄). After filtration, the solvent was removed in vacuo and the residue purified by column chromatography on silica gel. Elution with EtOAc/light petroleum (1:9) afforded the title compound 11.2 (130 mg, 0.0734 mmol, 70%) as an oil. [α]_{D}²⁰=−15.0 (c 2.0, CHCl₃). ¹H NMR (300 MHz, CDCl₃) δ 7.34-7.12 (m, 45H), 5.17-5.13 (m, 2H), 5.08-4.70 (m, 10H), 4.66-4.57 (m, 4H), 4.49-4.39 (m, 3H), 4.31-4.20 (m, 2H), 4.12-3.77 (m, 8H), 3.59-3.27 (m, 9H), 2.24-2.18 (m, 2H), 1.57-1.46 (m, 6H), 1.39-1.25 (m, 48H), 0.89 (t, J.6.3 Hz, 6H). ¹³C (75 MHz, CDCl₃) δ 173.3, 138.8, 138.6, 128.2, 127.9, 127.8, 127.4, 127.3, 101.5, 83.4, 82.6, 81.3, 81.2, 80.0, 75.8, 74.5, 74.7, 74.6, 73.3, 73.1, 72.8, 70.6, 69.3, 68.5, 63.3, 62.6, 34.1, 31.9, 30.1, 29.7, 29.5, 29.4, 26.1, 29.9, 24.9, 22.7, 14.1. ³¹P NMR (121.5 MHz, CDCl₃) δ 0.45, 0.00. HRMS-ESI [M+Na]⁺ calcd for C₁₁₀H₁₄₅O₁₇NaP: 1792.0117. Found 1792.0115.

α-D-Galactopyranosyl-1-O-(1-O-hexadecanoyl-2-O-hexadecyl-sn-glycero-3-phosphoryl)-glycerol (11.3). Pd(OH)₂/C (20%, 80 mg) was added to a stirred solution of the fully substituted 11.2 (100 mg, 0.0565 mmol) in THF/MeOH(2:3, 5 mL). The mixture was stirred under hydrogen for 2.5 h at rt and the hydrogen was replaced with argon. The mixture was filtered through Celite and the filtrate concentrated in vacuo. The residue was purified by column chromatography on silica gel. Elution with CHCl₃/CH₃OH/H₂O, (70:40:6) afforded the title compound 11.3, which was then lyophilized, to afford 11.3 (31 mg, 0.032 mmol, 54%) as a white powder. [α]_{D}²⁰=−2.72 (c 1.4, CHCl₃/CH₃OH/H₂O, 70:40:6). ¹H NMR (300 MHz, CDCl₃/CD₃OD/D₂O, 70:40:6) δ), 4.50-3.20 (m, 20H), 2.35 (t, J='7.8 Hz, 2H), 1.62-1.53 (m, 4H), 1.31-1.27 (m, 50H), 0.88 (t, J=6.9 Hz, 6H). ¹³C (75 MHz, CDCl₃) δ 176.05, 106.3, 82.2, 77.3, 76.2, 75,7, 74.9, 74.3, 73.3, 73.1, 72.1, 71.0, 65.9, 65.4, 63.3, 35.6, 33.2, 31.2, 31.0, 30.5, 27.4, 26.3, 23.9, 15.2. ³¹P NMR (121.5 MHz, CDCl₃/CD₃OD/D₂O, 70:40:6) δ 0.36. HRMS-ESI [M−H]⁻ calcd for C₄₇H₉₁O₁₇P: 957.5916. Found 957.5922. Anal. Calcd for C₄₇H₉₁O₁₇P.5H₂O: C, 53.80; H, 9.70. Found: C, 53.76; H, 9.20.

### Example 12

### Isolation of Bovine Dendritic Cells

Peripheral blood mononuclear cells (PBMCs) were isolated from heparinized blood of cattle by density-gradient centrifugation. Cells were adjusted to 10⁷/ml in RPMI-1640 medium containing 10% heat-inactivated FCS, 10 mM HEPES, 5×10⁻⁵ M 2-mercaptoethanol. Non-adherent cells were removed after 3 hours by washing with warm phosphate buffered saline (PBS). Adherent cells were then incubated in complete medium with 0.2 U/mI of recombinant bovine GM-CSF and 200 U/ml of recombinant bovine IL-4 (Both from Serotec, Oxford, UK), replenished daily. After 3-4 days, fresh media and cytokines were added to cells. Cells were harvested after 7-10 days of culture, washed three times and adjusted to desired cell numbers. When harvested, cells were more than 95% DCs based on the following: DCs lacked B and T cells markers had high expression of MHC class 2 and class 1 (Serotec), and had low expression of CD11a (Serotec) and CD14 (Serotec), and expressed CD11c (Veterinary Medical Research and Development, Inc. (VMRD, Pullman, WA, USA)). DCs after 10 days of culture also expressed CD80, CD86 and CD172a (Veterinary Medical Research and Development, Inc. (VMRD, Pullman, Wash., USA)). In addition, DCs had the characteristic veiled morphology and functional phenotype of DCs. DCs were used immediately by transferring to a 96-well microtiter plates at 2×10⁵ cells per 200 μL of complete medium. The cells were cultured in RPMI-1640 with 10% FCS, 10 mM HEPES and 4 mM L-glutamine, with the indicated concentrations of reagents, 50 pg of compounds 1.9 and 2.5, or positive control LPS, for 48 hours prior to measurements of IL-12 (FIG. 1).

### Example 13

### Isolation of Mouse Dendritic Cells

Bone marrow contents of C57BU6 mice were obtained from the femurs, by flushing the inside of the bones with media (see below). Cells were then incubated in complete medium with 0.2 U/mI of recombinant GM-CSF and 200 U/ml of recombinant IL-4 (Both from Serotec, Oxford, UK), replenished daily. After 3-4 days, fresh media and cytokines were added to cells. Cells were harvested after 7-10 days of culture, washed three times and adjusted to desired cell numbers. When harvested, cells were more than 95% DCs based on the following: DCs lacked B and T cells markers had high expression of MHC class 2 and class 1 (Serotec), and had low expression of CD11a (Serotec) and CD14 (Serotec), and expressed CD11c (Serotec). DCs after 10 days of culture also expressed CD80, CD86 and CD172a In addition, DCs had the characteristic veiled morphology and functional phenotype of DCs. DCs were used immediately by transferring to a 96-well microtiter plates at 2×10⁵ cells per 200 μL of complete medium. The cells were cultured in RPMI-1640 with 10% FCS, 10 mM HEPES and 4 mM L-glutamine in 5% CO₂/37° C., with the indicated concentrations of reagents, 50 μg of compounds 1.9, 2.5, 3.5, 4.6, 5.3, 6.2, 7.10, 8.6, 9.2, 10.3, PIM2 (Ainge G. D., Parlane N. A., Denis M., supra), and positive controls PAMCSK or MPL, for 48 hours prior to measurements of IL-12 and IL-10 (FIG. 2).

### Example 14

### IL-12 ELISA

Plates (Maxisorp, Nunc, Denmark) were coated with 8 μg/ml anti-IL-12 (B @ D Pharmaceuticals) and incubated overnight at room temperature. The plates were washed in washing buffer and blocking buffer was added for 1 h. Following a further washing step, samples were added for 1 h. Dilutions of the samples were added to the plates for 1 h. Following washing, biotin-labelled anti-IL-12 (Serotec: 8 pg/ml in blocking buffer) was added for 1 h, followed by washing and addition of SA-HRP for 45 min. Following the final washing step, TMB substrate was added, the reaction was stopped by the addition of H₂SO₄, and the absorbance values were read at 450 nm.

### Example 15

### IL-10 ELISA

Plates (Maxisorp, Nunc, Denmark) were coated with 8 μg/ml anti-IL-10 (B @ D Pharmaceuticals) and incubated overnight at room temperature. The plates were washed in washing buffer and blocking buffer was added for 1 h. Following a further washing step, samples were added for 1 h. Dilutions of the samples were added to the plates for 1 h. Following washing, biotin-labelled anti-IL-10 (B @ D Pharmaceuticals: 8 μg/ml in blocking buffer) was added for 1 h, followed by washing and addition of SA-HRP for 45 min. Following the final washing step, TMB substrate was added, the reaction was stopped by the addition of H₂SO₄, and the absorbance values were read at 450 nm.

### Example 16

### Analysis of Compounds

Th1 inducing activities of compounds 1.9 and 2.5 were tested in an in vitro bovine dendritic cell (DC) assay (FIG. 1). Products which direct the immune responses towards a Th1 profile are IL-12 inducers, and specialized antigen presenting cells such as DCs are excellent bioprobes for the secretion and release of these factors. Surprisingly, compound 1.9 induced very high levels of IL-12 from bovine dendritic cells, suggesting that this compound is a very good adjuvant candidate. Compound 2.5 was also a good adjuvant candidate inducing IL-12 levels similar to the natural compound PIM2. Supernatants were collected 48 hours after stimulation, and IL-12 levels measured. Columns with different letters are significantly different from each other (P<0.05).

IL-10 and IL-12 inducing activities of compounds 1.9, 2.5, 3.5, 4,6, 5.3, 6.2, 7.10, 8.6, 9.2, and 10.3 were tested in an in vitro mouse dendritic cell (DC) assay (FIG. 2). Cytokines which direct the immune responses towards a Thl profile are IL-12 inducers, and specialized antigen presenting cells such as DCs are excellent bioprobes for the secretion and release of these factors. On the other hand, IL-10 is an anti-inflammatory cytokine which diminishes the release of IL-12, and the balance in the production of these factors will determine the direction of the immune response. Products with adjuvant activities for cell-mediated immune responses are expected to stimulate IL-12, but IL-10 release prevents the adjuvants from having highly toxic side-effects associated with an unchecked release of IL-12. Surprisingly, all compounds induced high levels of IL-12 from mouse dendritic cells, suggesting that this class of compounds are very good adjuvant candidates. Perhaps even more surprising, all compounds induced IL-12 levels similar to or greater than the natural compound PIM2. Compounds 1.9 and 4.6 were the most potent inducing more IL-12 than the positive controls MPL and PAMCSK. All compounds induced some IL-10 but in lower amounts compared to IL-12. In summary the IL-12/IL-10 cytokine profile for this class of compound appeared similar to the positive control PAMCSK. Supernatants were collected 48 hours after stimulation, and IL-12 and IL-10 levels measured.

Where the foregoing description reference has been made to integers having known equivalents thereof, those equivalents are herein incorporated as if individually set forth.

Although the invention has been described in connection with specific preferred embodiments, it should be understood that the invention as claimed should not be unduly limited to such specific embodiments.

It is appreciated that further modifications may be made to the invention as described herein without departing from the spirit and scope of the invention.

### INDUSTRIAL APPLICABILITY

The invention relates to synthetic compounds that can, among other things, activate IL-10 and IL-12 secretion. The compounds are therefore useful in the treatment of diseases or conditions in which the secretion of IL-10 or IL-12 is desirable. Such diseases or conditions include infections, atopic disorders, and cancer. The compounds are also useful as adjuvants in the administration of vaccines.

1. A compound of the formula (I): 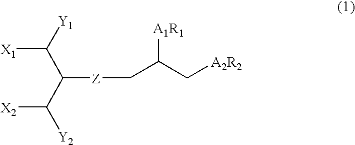
where:
X₁ and X₂ are H, or taken together form a 6-membered carbocyclic ring which is optionally substituted with one or more groups selected from the group consisting of OH, halogen, alkyloxy, acvloxy, and NH₂;
Y₁ and Y₂ are independently H, OH, or a saccharide having 1 to 5 glycosyl or glycosyloxy units, where one or more hydroxyl groups of one or more of the glycosyl or glycosyloxy units is optionally replaced with one or more groups selected from the group consisting of alkyloxy and acyloxy, provided that Y₁, and Y₂ are not both H;
Z is —O—(CH₂)ₙ₌₁₋₆—O—, —O—C(═O)—O—, —NH—C(═O)—O—, —O—C(═O)—NH—, —O—P(OH)(═O)—O—, —CH₂—P(—OH)(═O)—O—, —O—P(OH)(═O)—CH₂—, —O—P(OH)(═S)—O—, —CH₂—P(—OH)(═S)—O—, —O—P(—OH)(═S)—CH₂—, —CHF—P(—OH)(═O)—O—, —O—P(—OH)(═O)—CF₂, or —0—P(—OH)(═O)—CHF—;
A₁ and A₂ are independently selected from the group consisting of O, NH, CH₂, CHF, and CF₂; and
R₁ and R₂ are independently linear or branched alkyl or acyl groups having up to 30 carbon atoms, which may be saturated or may be unsaturated having up to 4 units of unsaturation, and provided that R₁ and R₂ are not both acyl;
or a pharmaceutically acceptable salt or hydrate thereof.

2. A compound as claimed in claim 1 where X₁ and X₂ are both H.

3. A compound as claimed in claim 1 where X₁ and X₂ taken together form a 6-membered carbocyclic ring.

4. A compound as claimed in claim 3 where the 6-membered carbocyclic ring formed by X₁ and X₂ is an inositol.

5. A compound as claimed in claim 4 where one or more of the secondary hydroxyl groups of the inositol are replaced with alkyloxy or acyloxy groups.

6. (canceled)

7. (canceled)

8. A compound as claimed in claim 4 where inositol is D-myo-inositol and the 3-hydroxyl group is optionally replaced with an alkyloxy group or an acyloxy group.

9.-12. (canceled)

13. A compound as claimed in claim 1 where at least one of Y₁ and Y₂ is a saccharide having 1 to 5 glycosyl or glycosyloxy units.

14. (canceled)

15. A compound as claimed in claim 13 where the glycosyl or glycosyloxy units of each saccharide are attached by 1-6 α glycosidic linkages, 1-2 α glycosidic linkages, 1-6 β glycosidic linkages, 1-2 β glycosidic linkages, or combinations thereof.

16. (canceled)

17. A compound as claimed in claim 1 where the glycosyl or glycosyloxy units are each independently selected from the group consisting of mannosyl, mannosyloxy, galactosyl, galactosyloxy, glucosyl, glucosyloxy, glucosaminyl, and glucosaminyloxy.

18. A compound as claimed in claim 17 where the glycosyl or glycosyloxy units are D-isomers.

19. A compound as claimed in claim 15 where one or more of the hydroxyl groups of one or more of the glycosyl or glycosyloxy units are replaced with an alkyloxy group or an acyloxy group.

20.-23. (canceled)

24. A compound as claimed in claim 1 having the formula (2): 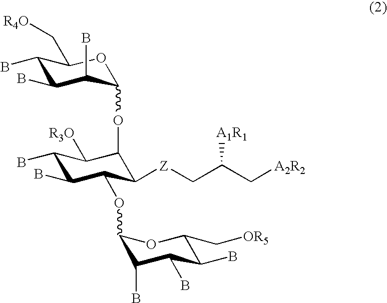
where:
R₃ and R₄ are each independently H, or linear or branched alkyl or acyl groups having up to 30 carbon atoms, which may be saturated or may be unsaturated having up to 4 units of unsaturation;
R₅ is H or a saccharide having 1 to 4 glycosyl or glycosyloxy units, where each glycosyl or glycosyloxy unit is selected from the group consisting of mannosyl, mannosyloxy, galactosyl, galactosyloxy, glucosyl, glucosyloxy, glucosaminyl, and glucosaminyloxy;
or R₅ is a radical of formula: 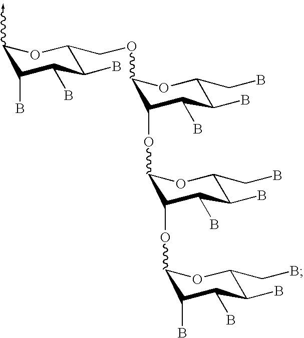
each B is independently H or OH; and
where the compound can include α glycosidic linkages, β glycosidic linkages or both α and β glycosidic linkages.

25. A compound as claimed in claim 1 selected from the group consisting of:

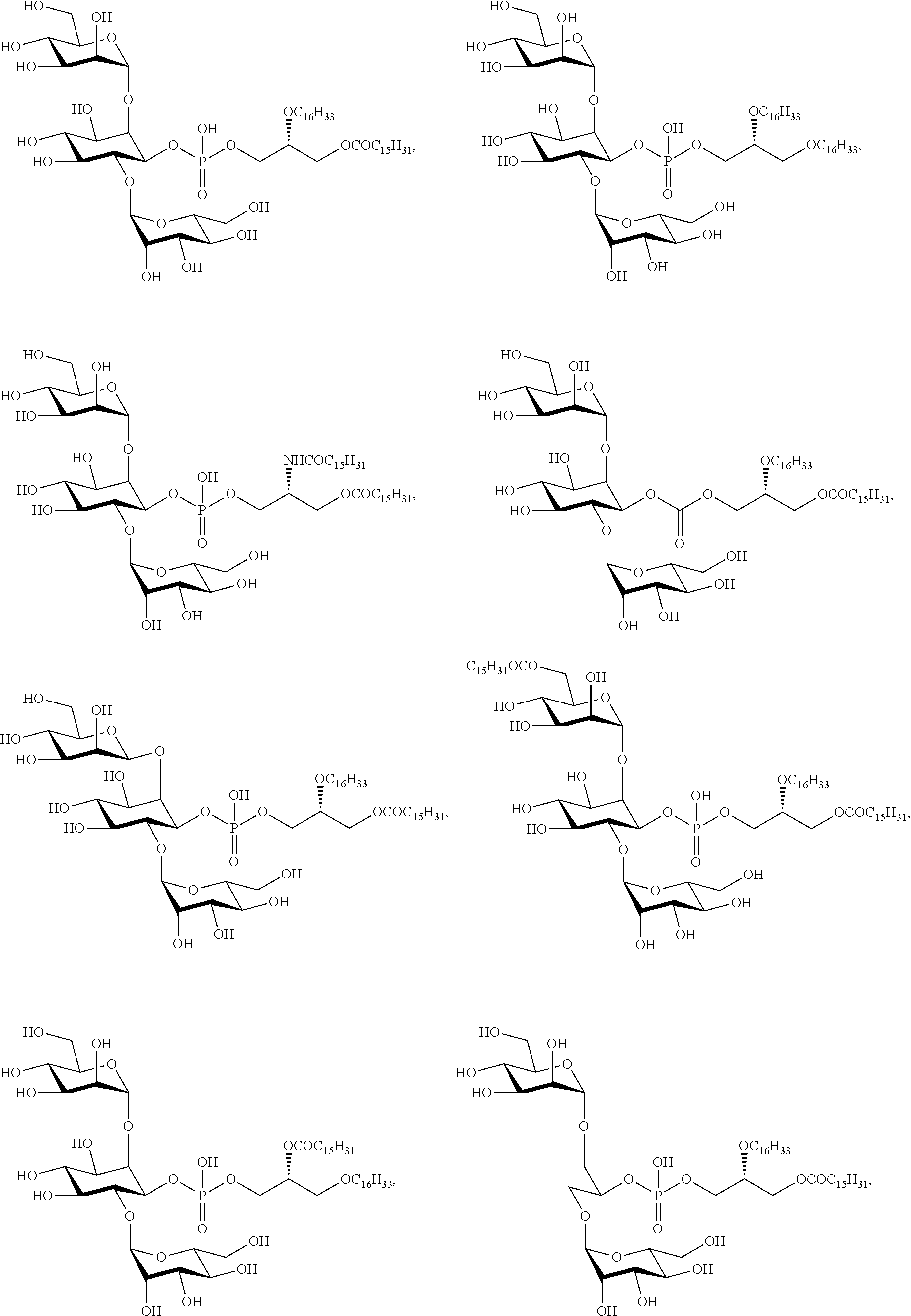

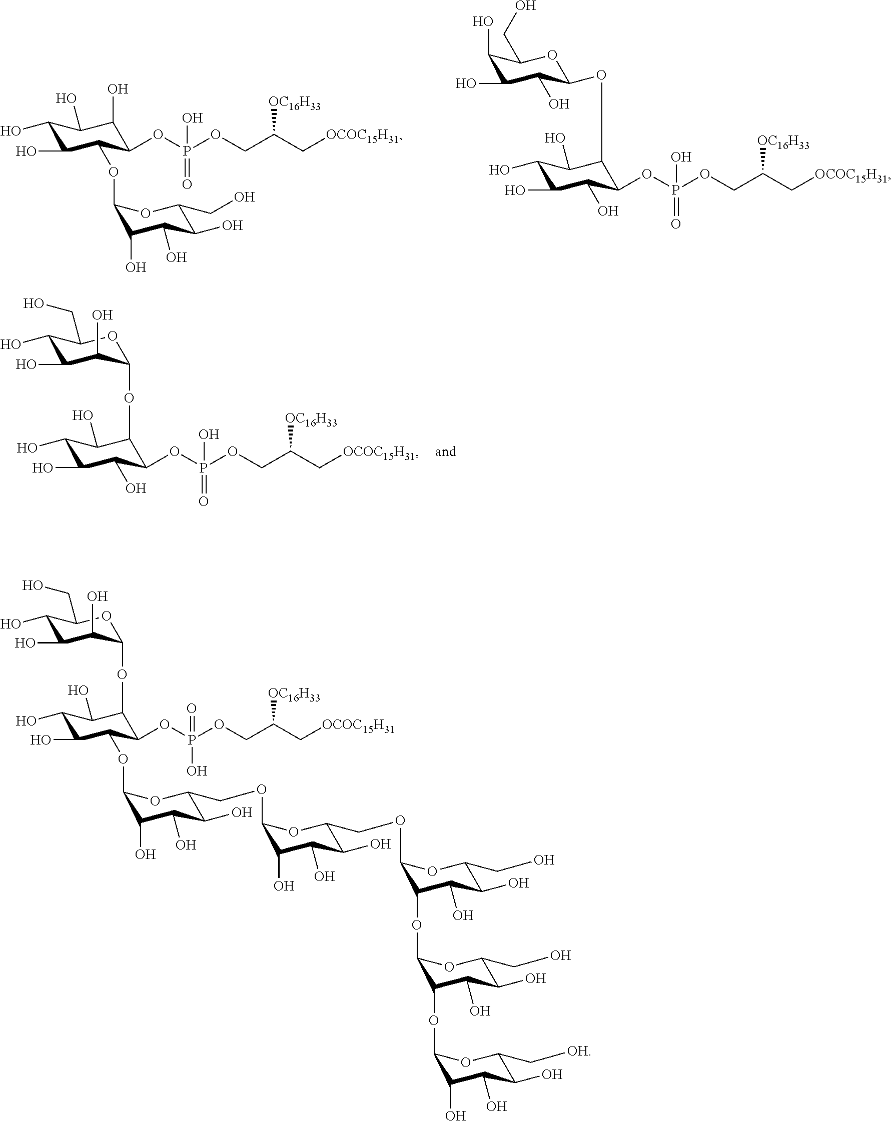

26. A compound as claimed in claim 1 having the formula (3): 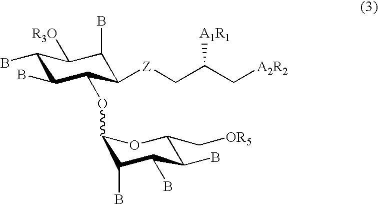
where B is independently H or OH;
R₃ is H, or linear or branched alkyl or acyl groups haying up to 30 carbon atoms, which may be saturated or may be unsaturated having up to 4 units of unsaturation;
R₅ is H or a saccharide haying 1 to 4 glycosyl or glycosyloxy units, where each glycosyl or glycosyloxy unit is selected from the group consisting of mannosyl, mannosyloxy, galactosyl, galactosyloxy, glucosyl, glucosyloxy, glucosaminyl, and glucosaminyloxy;
or R₅ is a radical of formula: 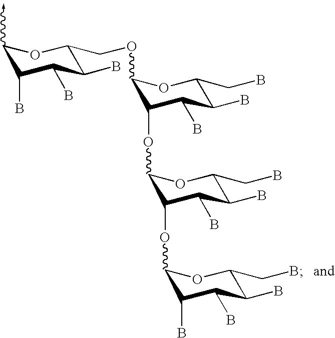
the compound can include a glycosidic linkages, β glycosidic linkages, or both α and β glycosidic linkages.

27. (canceled)

28. A compound as claimed in claim 1 having the formula (4): 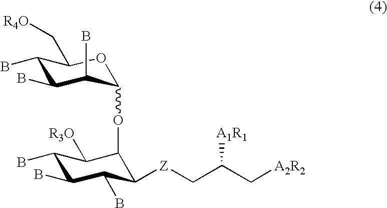
where
B is independently H or OH;
R₃ is H, or linear or branched alkyl or acyl groups having up to 30 carbon atoms, which may be saturated or may be unsaturated having up to 4 units of unsaturation;
R₅ is H or a saccharide having 1 to 4 glycosyl or glycosyloxy units, where each glycosyl or glycosyloxy unit is selected from the group consisting of mannosyl, mannosyloxy, galactosyl, galactosyloxy, glucosyl, glucosyloxy, glucosaminyl, and glucosaminyloxy;
or R₅ is a radical of formula: 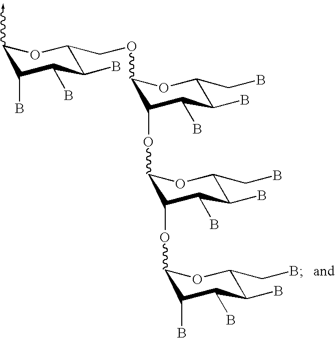
the compound can include a glycosidic linkages, β glycosidic linkages, or both α and β glycosidic linkages.

29.-37. (canceled)

38. A compound as claimed in claim 24 where R₅ is H.

39. A compound as claimed in claim 24 where A₁ and A₂ are O.

40. A compound as claimed in claim 24 where each B is OH.

41. A pharmaceutical composition containing a compound of claim 1, or a pharmaceutically acceptable salt or hydrate thereof, and one or more pharmaceutically acceptable carriers, excipients, or diluents.

42. A pharmaceutical composition as claimed in claim 41 where the compound of claim 1 is an adjuvant in admixture with a therapeutic or preventative agent.

43. A pharmaceutical composition as claimed in claim 42 where the therapeutic or preventative agent is a vaccine.

44. (canceled)

45. A method of treating or preventing infection, an atopic disorder, or cancer comprising administering to a patient an effective amount of a compound of claim 1.

46. A method as claimed in claim 45 where the infection, atopic disorder, or cancer is any one of pneumonia, bacteraemia, bacterial meningitis, bacterial peritonitis, urethritis, cervicitis, proctitis, pharyngitis, salpingitis, epididymitis, gastroenteritis, enteric fever, bacillary dysentery, tetanus, ghonorhea, syphilis, toxic shock syndrome, arthritis, impetigo, infective endocarditis, focal infection, pleural empyema, pleural effusion, tuberculosis, contact dermatitis, atopic dermatitis, seborrheic dermatitis, nummular dermatitis, chronic dermatitis of the hands and feet, generalized exfoliative dermatitis, stasis dermatitis, lichen simplex chronicus, acute rhinitis, allergic rhinitis, chronic rhinitis, atrophic rhinitis, vasomotor rhinitis, hay fever, perennial rhinitis, allergic conjunctivitis, sinusitis, urticaria, uveitis, food allergy, anaphylaxis, mastocytosis, hives, hypersensitivity pneumonitis, eosinophilic pneumonias, allergic bronchopulmonary aspergillosis, giant bullae, bronchitis, bronchiospasm, emphysema, asthma, chronic obstructive pulmonary disease, bladder cancer, melanoma, non-melanoma skin cancer, breast cancer, colon cancer, rectal cancer, pancreatic cancer, endometrial cancer, prostate cancer, kidney (renal cell) cancer, thyroid cancer, lung cancer, leukaemia, non-Hodgkin's lymphoma, squamous cell carcinoma, adenocarcinoma, basal cell carcinoma, large cell carcinoma, renal cell carcinoma, hepatocellular carcinoma, osteosarcoma, fibrosarcoma, neuroblastoma, glioma, astrocytoma, medulloblastoma, adenoma, lymphoma, and myeloma.

47.-49. (canceled)

50. A compound as claimed in claim 26 where R₅ is H.

51. A compound as claimed in claim 28 where R₅ is H.

52. A compound as claimed in claim 26 where A₁ and A₂ are O.

53. A compound as claimed in claim 28 where A₁ and A₂ are O.

54. A compound as claimed in claim 26 where each B is OH.

55. A compound as claimed in claim 28 where each B is OH.
